(19) United States
(12) Patent Application Publication
(10) Pub. No.: US 2016/0311812 A1
(43) Pub. Date: Oct. 27, 2016

(54) ADENOSINE A1 AGONISTS AS MEDICAMENTS FOR KIDNEY DISORDERS

(71) Applicant: BAYER PHARMA AKTIENGESELLSCHAFT, Berlin (DE)

(72) Inventors: Barbara ALBRECHT-KÜPPER, Wülfrath (DE); Kirsten LEINEWEBER, Velbert-Langenberg (DE); Axel KRETSCHMER, Wuppertal (DE); Daniel MEIBOM, Wuppertal (DE); Alexandros VAKALOPOULOS, Hilden (DE); Nicole DIEDRICHS, Velbert (DE); Katja ZIMMERMANN, Düsseldorf (DE)

(73) Assignee: Bayer Pharma Aktiengesellschaft, Berlin (DE)

(21) Appl. No.: 15/103,307

(22) PCT Filed: Dec. 9, 2014
(86) PCT No.: PCT/EP2014/076978

§ 371 Date: Jun. 9, 2016

(30) Foreign Application Priority Data
Dec. 12, 2013 (EP) 13196780.4

(51) Int. Cl. C07D 417/12; C07C 53/18; C07D 417/14

(52) U.S. Cl. CPC C07D 417/12; C07D 417/14; C07C 53/18

(57) ABSTRACT

The present application relates to selective partial adenosine A1 receptor agonists of the formula (I) and their use for treating and/or preventing diseases and to their use for preparing medicaments for treating and/or preventing diseases, preferably for treating and/or preventing acute and/or chronic kidney disorders (primary disorder and secondary disorder) with and without concomitant acute and/or chronic heart disorders.

The present application relates to selective partial adenosine A1 receptor agonists of the formula (I) and their use for treating and/or preventing diseases and to their use for preparing medicaments for treating and/or preventing diseases, preferably for treating and/or preventing acute and/or chronic kidney disorders (primary disorder and secondary disorder) with and without concomitant acute and/or chronic heart disorders.

Furthermore, the present application relates to selective partial adenosine A1 agonists of the formula (I) and their use for treating and/or preventing diseases and to their use for preparing medicaments for treating and/or preventing diseases, preferably for treating and/or preventing chronic kidney disorders with and without concomitant acute and/or chronic heart disorders.

The kidneys represent an important regulatory system in the body for detoxification (excretion of metabolic waste products usually eliminated in the urine, including creatinine, urea and uric acid), electrolyte metabolism (such as sodium, calcium, potassium and phosphorus), regulation of blood pH and of fluid balance, regulation of the blood pressure (including by volume regulation, modulation of the renin-angiotensin-aldosterone system), for hormone production (including erythropoietin and vitamin D) and for bone metabolism. Kidney failure therefore has far-reaching consequences for the whole organism.

Here, kidney failure or kidney diseases can be differentiated into acute, reversible and chronic, irreversible forms, independently of whether it is a primary or secondary form of the disorder (co-morbidity). Depending on the trigger, duration and severity of acute kidney failure or of acute kidney disease, manifestation as chronic kidney failure or chronic kidney disease is probable.

The commonest trigger for acute kidney failure or kidney diseases is renal hypoperfusion, e.g. as a result of acute volume loss (including blood loss, fluid volume deficit), a drop in blood pressure with reduced renal perfusion pressure (e.g. in the context of acute and/or congestive heart failure) and/or occlusion of the renal vessels (including through stenoses and/or thrombi and/or embolisms in the renal arteries and/or veins). Furthermore, inflammatory diseases (glomerulonephritis), medication at high dosage and/or long duration such as antibiotics, nonsteroidal anti-inflammatory drugs (NSAIDs) and cytostatics, as well as heavy metals, alcohol and X-ray contrast media lead to acute kidney injury.

The commonest triggers for chronic kidney failure or kidney diseases (permanent, slowly progressing loss of function of the kidneys over the course of months and years), in addition to acute renal failure or acute kidney disease, include long-standing diabetes mellitus, high blood pressure (arterial hypertension), nephritides (glomemlonephritis), repeated inflammations of the renal pelvis (pyelonephritis), hypovolaemia, renal artery stenosis, hepatorenal syndrome and/or heart failure.

The functionally smallest unit in the kidney is the nephron, consisting of glomerulus/Bowman's capsule, the proximal tubule, the loop of Henle, the distal tubule and the collecting duct, wherein in all these segments of the nephron, various transport and diffusion processes take place, which serve for the filtration, reabsorption and secretion of substances that are usually eliminated in the urine, ions and water. Filtration takes place in the glomemli, the glomerular filtration rate (GFR) being regulated by the vascular resistance (=filtration pressure) of the vas afferens (afferent arteriole of the glomemlus) and of the vas efferens (efferent arteriole of the glomemlus). Myogenic mesangial cells in the matrix of the glomerulus are in direct contact with the glomerular basal membrane and affect, via their contraction or relaxation, the capillary bed of the glomemlus and therefore the GFR. However, mesangial cells not only play a physiological role in regulation of the capillary bed but also an essential role in the pathophysiology of kidney disorders, as their malfunction is accompanied by the release of pro-inflammatory, pro-proliferative and pro-fibrotic factors, which depending on type, duration and extent can have a decisive influence on the development of functional and/or structural, reversible and/or irreversible kidney injury, such as in the case of glomerulonephritis and/or glomerulosclerosis.

Adenosine, a purine nucleoside, is present in all cells and is released under a large number of physiological and pathophysiological stimuli Adenosine is formed intracellularly in the degradation of adenosine-5′-monophosphate (AMP) and S-adenosylhomocysteine as intermediate, but can be released from the cell and then, by binding to specific receptors, exerts functions as hormone-like substance or neurotransmitter.

The action of adenosine is mediated via specific receptors. To date, the subtypes A1, A2a, A2b and A3 are known. “Adenosine receptor-selective ligands” means, according to the invention, those substances that bind selectively to one or more subtypes of the adenosine receptors and either imitate the action of adenosine (adenosine receptor agonists) or block its action (adenosine receptor antagonists).

The effects of these adenosine receptors are mediated intracellularly by the messenger cAMP. In the case of binding of adenosine to the A2a or A2b receptors, through activation of the membrane-bound adenylate cyclase there is an increase in the intracellular cAMP concentration, whereas binding of adenosine to the A1 or A3 receptors causes, through inhibition of adenylate cyclase, a decrease in the intracellular cAMP concentration.

In the cardiovascular system, activation of A1 receptors by specific A1 agonists leads to a rate-dependent lowering of the heart rate, negative inotropism and protection of the heart against ischaemia (“pre-conditioning”) without affecting the systemic blood pressure. Selective A1 agonists might therefore be suitable inter alia for the treatment of angina pectoris and atrial fibrillation (see the review article, I. Giorgi, P. Nieri, Expert. Opin. Ther. Patents 2008, 18: 677-691) and, based on demonstrated cardioprotective properties, could be used for treatment and organ protection in acute myocardial infarction, acute coronary syndrome, cardiac insufficiency, bypass operations, cardiac catheterization and organ transplants (K. Zimmermann et al. Clin Res Cardiol 2011, 100 (Suppl. 1) P1692; B. Albrecht-Kiipper et al. Purinergic Signalling 2012, (Suppl. 1): S91-S99).

Apart from the heart, A1 receptors are, moreover, also expressed in the kidney, in particular in the renal cortex, in the boundary between the renal cortex and the renal medulla (the corticomedullary boundary) and in the internal renal medulla (Vizthum et al., Kidney Internat. 2004, 65, 1180-1190) and here they regulate preglomemlar vasoconstriction (vas afferens), tubuloglomerular feedback (TGF), the release of renin, erythropoietin and catecholamines from sympathetic nerve endings, and sodium reabsorption and the release of aldosterone.

The content of the endogenous A1 receptor agonist adenosine in the kidney is subject to marked fluctuations or a tissue-specific distribution: generally for adenosine, under normoxic conditions, the concentration of free adenosine in the extracellular space is very low, whereas areas with low oxygen supply have a higher content of adenosine. This applies in particular in the kidney, in which the renal medulla has far lower oxygen supply, compared with the renal cortex. Therefore even under physiological conditions the adenosine concentration in the renal medulla per se is 3-4 times higher than the adenosine concentration in the renal cortex.

Considered physiologically (e.g. in the renal medulla) or pathophysiologically (e.g. in the renal cortex in ischaemic/hypoxic conditions), in less well perfused areas there is generally a mismatch between the work to be performed by the cells (oxygen consumption) and perfusion, i.e. between supply with oxygen (oxygen supply)/nutrients and demand. And in fact the action of adenosine consists of increasing the oxygen supply to the affected areas or throttling-back the metabolism of these areas, so as to adapt to ischaemic or hypoxic conditions.

With reference to oxygen supply, it has been shown that continuous infusion with adenosine leads to a redistribution of cortical perfusion through vasoconstriction of the vas afferens from the outer layers into the inner regions of the corticomedullary boundary near the renal medulla (see Spielmann W S, Britton S L and Fiksen-Olsen M J (1980) Effect of adenosine on the distribution of renal blood flow in dogs. Circ Res 46:449-456; Macias J F, Fiksen-Olsen M J, Romero J C and Knox F G (1983) Intrarenal blood flow distribution during adenosine mediated vasoconstriction. Am J Physiol 244:H138-H141). The resultant reduced perfusion of the outer renal cortical region is accompanied by reduced diuresis and natriuresis, as well as reduced creatinine clearance, i.e. a reduced GFR, relative to these parameters, and thus nominally by a decrease in renal function. However, stimulation of the A1 receptors in the kidney also has an inhibitory effect on the release of renin and aldosterone, and an intensifying effect on the release of erythropoietin. Whereas the former contributes to reduced activation of the RAAS and therefore to a decrease of angiotensin II-mediated functional and structural maladaptive remodeling in the kidney, the latter leads to increased formation of red blood cells, therefore to improved oxygen loading of the blood and therefore to improved oxygen supply of the whole organism. In this connection it should be pointed out that in ischaemic kidney injury, A1 agonists continue to have structurally protective action through anti-inflammatory and antifibrotic effects and thus protect the kidney against tissue damage and the resultant functional disturbances. Thus, it was shown in mice that stimulation of A1 receptors protects the kidney against ischaemia-reperfusion-induced tubular apoptosis, necrosis and inflammation in the outer renal medulla along the corticomedullary boundary (H. T. Lee et al., J. Am. Soc. Nephrol. 2004, 15, 102-111), whereas through blockade of the A1 receptors with A1 receptor antagonists or in A1 receptor knock-out mice there is increased occurrence of ischaemia-reperfusion-induced structural renal damage (M. Kim et al., Kidney Int. 2009, 75, 809-823).

Thus, whereas acute stimulation of the A1 receptors in the kidney depresses the functionality of the nephrons (diuresis, natriuresis, creatinine clearance, GFR), stimulation of the A1 receptors is nevertheless necessary to prevent or suppress any mismatch in oxygen supply and oxygen demand, as well as structural changes of the kidney through ischaemia-induced pro-inflammatory and pro-fibrotic processes.

Accordingly, both the chronic use of a full A1 receptor agonist (reduced functionality of the nephron as a result of reduced cortical perfusion) and the chronic use of a full A1 receptor antagonist (absence of protection against ischaemia-reperfusion-induced pro-inflammatory and pro-fibrotic processes and structural damage) are limited in acute and/or chronic kidney disorders.

It was found, surprisingly, that selective partial A1 receptor agonists of formula (I) are able to provide functional and structural protection of the kidney, without causing a decrease in renal function (such as diuresis, natriuresis, creatinine clearance, GFR).

The extent of agonistic action is stated as intrinsic activity, with values between 0 and 1. Whereas a full agonist is characterized by a maximum intrinsic activity of 1 and a full antagonist by absence of intrinsic activity, thus 0, the intrinsic activity of a partial agonist can be between >0 and <1. Whereas agonists with an intrinsic activity displace the receptor equilibrium so that almost all receptors assume an active conformation, a full (neutral) antagonist with an intrinsic activity of 0 does not alter the initial receptor equilibrium. In contrast, a partial agonist only displaces some of the receptors into the active conformation, in accordance with its intrinsic activity between >0 and <1. The actual action profile of a partial agonist consists, however, not only of the reduced strength, but rather that in the presence of a full agonist it acts as an antagonist.

Therefore, transferred to the kidney, if there is functional and structural acute and/or chronic kidney injury, associated with perfusion disturbances and/or ischemia and/or hypoxia, a partial A1 receptor agonist would, depending on the endogenous adenosine concentration, behave as a weak agonist and/or as an antagonist, simultaneously, but spatially separate from one another.

The ligands known from the prior art, regarded as “adenosine receptor-specific”, are mainly derivatives based on natural adenosine [S-A. Poulsen and R. J. Quinn, “Adenosine receptors: New opportunities for future drugs”, Bioorganic and Medicinal Chemistry 6 (1998), pages 619-641]. However, these adenosine ligands known from the prior art generally have the drawback that their action is not really receptor-specific, their action is weaker than that of natural adenosine, after oral application their action is very weak or they have undesirable side effects on the central nervous system (CNS) (A. K. Dhalla et al., Curr. Topics in Med. Chem. 2003, 3, 369-385; [E. Elzein, J. Zablocki, Exp. Opin. Invest. Drugs 2008, 17(12), 1901-1910). Therefore they are mainly only used for experimental purposes. Compounds of this kind in clinical development are currently only suitable for intravenous application.

Prodrugs are derivatives of an active substance, which in vivo go through a single-stage or multistage biotransformation of an enzymatic and/or chemical nature, before the actual active substance is released. A prodrug residue is used as a rule in order to improve the property profile of the underlying active substance [P. Ettmayer et al., J. Med. Chem. 47, 2393-2404 (2004)]. To achieve an optimal action profile, both the design of the prodrug residue and the desired release mechanism must be tailored very precisely to the individual active substance, the indication, the site of action and the route of administration. A great many medicinal products are administered as prodrugs, which have improved bioavailability relative to the underlying active substance, achieved for example through improvement of the physicochemical profile, especially the solubility, the active or passive absorption properties or the tissue-specific distribution. From the extensive literature on prodrugs, we may for example mention: H. Bundgaard (ed.), Design of Prodrugs: Bioreversible derivatives for various functional groups and chemical entities, Elsevier Science Publishers B.V., 1985. A review of prodrug derivatives based on carboxylic acid esters and possible properties of such compounds is given, for example, in K. Beaumont et al., Curr. Drug Metab. 4, 461-485 (2003). Furthermore, dipeptide prodrugs of acyclovir are known for the treatment of herpes infections of the eyes (B. S. Anand et al., Curr. Eye Res. 26, No. 3-4, 151-163 (2003)), which prodrugs address the oligopeptide transporter on the cornea, to increase the bioavailability of acyclovir in the eye.

WO 01/25210, WO 02/070484, WO 02/070485, WO 2002/070520, WO 03/053441, WO 2008/028590, WO 2008/064789, WO 2009/100827, WO 2009/015776, WO 2009/015812, WO 2009/112155 and WO 2009/143992 disclose different substituted 3,5-dicyano-6-aminopyridines as adenosine receptor ligands for the treatment of cardiovascular disorders. WO 2006/027142 describes substituted phenylaminothiazoles, WO 2008/064788 describes cyclically substituted 3,5-dicyanopyridines, WO 2009/080197 discloses substituted azabicyclic adensonine receptor ligands, WO 2009/015811, WO 2009/015812, WO 2010/072314 and WO 2010/072315 describe amino acid ester prodrugs of 3,5-dicyano-6-aminopyridines. WO2010/086101 discloses further adenosine receptor ligands for the treatment of cardiovascular disorders. WO 03/053441 and WO 07/073855 (A1) describe selective A1 receptor agonists of the 2-thio-3,5-dicyano-4-phenyl-6-aminopyridine type in combination with aminoglycosides for protecting renal cells against antibiotics-induced renal cell damage. WO2009/015811 discloses prodrug derivatives of 2-amino-6-({[2-(4-chlorophenyl)-1,3-thiazol-4-yl]methyl}thio)-4-[4-(2-hydroxyethoxy)phenyl]pyridine-3,5-dicarbonitrile and inter alia their use for acute kidney failure and nephropathy. WO 10/086101 describes various alkylamino-substituted dicyanopyridines and their amino acid ester prodrugs and, in addition to their main use for cardiovascular disorders, also their use for kidney disorders. However, the document mentions neither specific kidney disorders or results with respect to their potential activity.

However, from the entire prior art, it was not known that partial agonists of the formula (I) can provide protection of the kidney in acute and/or chronic kidney disorders, as presented below, without negatively impacting renal function.

Accordingly, one object of the present invention is the provision and targeted selection of potent and selective partial A1 receptor agonists that have an advantageous therapeutic and/or pharmacologically dual action profile, and are suitable as such for treating and/or preventing acute functional and/or structural kidney disorders (primary disorder and secondary disorder).

Accordingly, a further object of the present invention is the provision and targeted selection of potent and selective partial A1 receptor agonists that have an advantageous therapeutic and/or pharmacologically dual action profile, and are suitable as such for treating and/or preventing chronic functional and/or structural kidney disorders (primary disorder and secondary disorder).

For the purpose of the present invention, the suitability for the treatment and/or prophylaxis of acute kidney disorders is to be understood as meaning in particular the suitability for the treatment and/or prophylaxis of acute renal insufficiency and of acute kidney failure (primary disorder and secondary disorder).

For the purpose of the present invention, the suitability for the treatment and/or prophylaxis of chronic kidney disorders is to be understood as meaning in particular the suitability for the treatment and/or prophylaxis of chronic renal insufficiency and of chronic kidney failure (primary disorder and secondary disorder).

In the context of the present invention, the term “acute renal insufficiency” encompasses acute manifestations of kidney disease, of kidney failure and/or renal insufficiency with and without the need for dialysis, and also underlying or related renal disorders such as renal hypoperfusion, intradialytic hypotension, volume deficiency (e.g. dehydration, blood loss), shock, acute glomerulonephritis, haemolytic-uraemic syndrome (HUS), vascular catastrophe (arterial or venous thrombosis or embolism), cholesterol embolism, acute Bence-Jones kidney in the event of plasmacytoma, acute supravesicular or subvesicular efflux obstructions, immunological renal disorders such as kidney transplant rejection, immune complex-induced renal disorders, tubular dilatation, hyperphosphataemia and/or acute renal disorders characterized by the need for dialysis. Moreover, in partial resections of the kidney, dehydration through forced diuresis, uncontrolled blood pressure increase with malignant hypertension, urinary tract obstruction and infection and amyloidosis and systemic diseases with glomerular involvement, such as rheumatologic-immunologic systemic diseases, for example lupus erythematosus, renal artery thrombosis, renal vein thrombosis, analgesic nephropathy and renal-tubular acidosis, and X-ray contrast media-induced and medication-induced acute interstitial kidney disorders.

In the context of the present invention, the term “chronic renal insufficiency” encompasses chronic manifestations of kidney disease, of kidney failure and/or renal insufficiency with and without the need for dialysis, and also underlying or related renal disorders such as renal hypoperfusion, intradialytic hypotension, obstructive uropathy, glomerulopathy, glomerular and tubular proteinuria, renal oedema, haematuria, primary, secondary and chronic glomemlonephritis, membranous and membranoproliferative glomemlonephritis, Alport syndrome, glomerulosclerosis, tubulointerstitial disorders, nephropathic disorders such as primary and congenital kidney disease, renal inflammation, immunological renal disorders such as kidney transplant rejection, immune complex-induced renal disorders, diabetic and non-diabetic nephropathy, pyelonephritis, renal cysts, nephrosclerosis, hypertensive nephrosclerosis and nephrotic syndrome, which can be characterized diagnostically, for example, by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, glomerular and arteriolar lesions, tubular dilatation, hyperphosphataemia and/or the need for dialysis. Moreover, chronic renal insufficiency in renal cell carcinomas, after partial resections of the kidney, dehydration through forced diuresis, uncontrolled blood pressure increase with malignant hypertension, urinary tract obstruction and infection and amyloidosis and systemic diseases with glomerular involvement, such as rheumatologic-immunologic systemic diseases, for example lupus erythematosus, and renal artery stenosis, renal artery thrombosis, renal vein thrombosis, analgesic nephropathy and renal-tubular acidosis are encompassed. In addition chronic renal insufficiency owing to X-ray contrast agent- and medicament-induced chronic interstitial renal disorders, metabolic syndrome and dyslipidaemia. The present invention also encompasses the use of the compounds of the invention for the treatment and/or prophylaxis of sequelae of renal insufficiency, for example pulmonary oedema, heart failure, uraemia, anaemia, electrolyte disorders (for example hyperkalaemia, hyponatraemia) and disorders in bone and carbohydrate metabolism.

For the purpose of the present invention, disorders of the cardiovascular system or cardiovascular disorders are to be understood as meaning, for example, the following disorders: hypertension, peripheral and cardiac vascular disorders, coronary heart disease, coronary restenosis such as, for example, restenosis after balloon dilatation of peripheral blood vessels, myocardial infarction, acute coronary syndrome, acute coronary syndrome with ST elevation, acute coronary syndrome without ST elevation, stable and unstable angina pectoris, myocardial insufficiency, Prinzmetal angina, persistent ischaemic dysfunction (“hibernating myocardium”), transient postischaemic dysfunction (“stunned myocardium”), heart failure, tachycardias, atrial tachycardia, arrhythmias, atrial and ventricular fibrillation, persistent atrial fibrillation, permanent atrial fibrillation, atrial fibrillation with normal left ventricular function, atrial fibrillation with impaired left ventricular function, Wolff-Parkinson-White syndrome, disturbances of peripheral blood flow, elevated levels of fibrinogen and of low density LDL, and elevated concentrations of plasminogen activator inhibitor 1 (PM-1).

For the purpose of the present invention, the term heart failure includes both acute and chronic manifestations of heart failure, as well as more specific or related types of disease, such as acute decompensated heart failure, right heart failure, left heart failure, global failure, ischaemic cardiomyopathy, dilated cardiomyopathy, congenital heart defects, heart valve defects, heart failure associated with heart valve defects, mitral stenosis, mitral valve insufficiency, aortic stenosis, aortic valve insufficiency, tricuspid stenosis, tricuspid insufficiency, pulmonary stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, diastolic and systolic heart failure (i.e. heart failure with preserved ejection fraction (HFpEF) and heart failure with reduced ejection fraction (HFrEF)).

Accordingly, the present invention provides for the use of compounds of the formula (I)

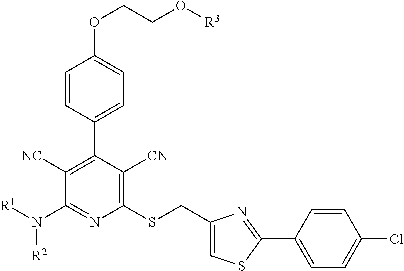

in which
R¹ represents hydrogen or (C₁-C₄)-alkyl,
R² represents hydrogen or (C₁-C₄)-alkyl,
   where (C₁-C₄)-alkyl may be substituted by 1 to 3 substituents independently of one another selected from the group consisting of fluorine, trifluoromethyl, trifluoromethoxy, (C₁-C₄)-alkoxy, (C₃-C₇)-cycloalkyl, (C₃-C₇)-cycloalkoxy and (C₁-C₄)-alkylsulphonyl,
or
R¹ and R² together with the nitrogen atom to which they are attached form a 4- to 7-membered heterocycle which may contain a further heteroatom from the group consisting of N, O and S,
   where the 4- to 7-membered heterocycle may be substituted by 1 to 2 substituents independently of one another selected from the group consisting of fluorine, trifluoromethoxy, (C₁-C₄)-alkyl, trifluoromethoxy and (C₁-C₄)-alkoxy,
R³ represents hydrogen or a group of the formula

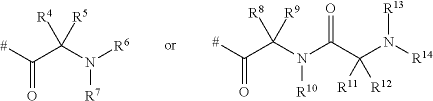

where
# represents the point of attachment to the oxygen atom,
R⁴ represents hydrogen or the side group of a natural α-amino acid or its homologues or isomers,
R⁵ represents hydrogen,
R⁶ represents hydrogen,
R⁷ represents hydrogen,
R⁸ represents hydrogen or the side group of a natural α-amino acid or its homologues or isomers,
R⁹ represents hydrogen,
R¹⁰ represents hydrogen or methyl,
R¹¹ represents hydrogen or the side group of a natural α-amino acid or its homologues or isomers,
R¹² represents hydrogen,
R¹³ represents hydrogen,
R¹⁴ represents hydrogen,
and the N-oxides, salts, solvates, salts of the N-oxides and solvates of the N-oxides or salts thereof in a method for the treatment and/or prevention of acute and/or chronic kidney disorders with or without concomittant acute and/or chronic heart disorders.

Accordingly, the present invention further provides for the use of compounds of the formula (I)

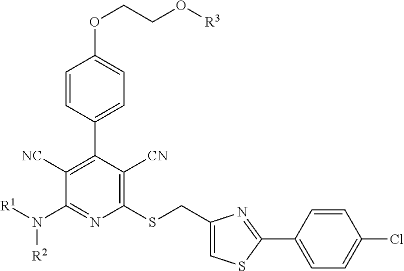

in which
R¹ represents (C₁-C₄)-alkyl,
R² represents (C₁-C₄)-alkyl,
   where (C₁-C₄)-alkyl may be substituted by 1 to 3 substituents independently of one another selected from the group consisting of fluorine, trifluoromethyl, trifluoromethoxy, (C₁-C₄)-alkoxy, (C₃-C₇)-cycloalkyl, (C₃-C₇)-cycloalkoxy and (C₁-C₄)-alkylsulphonyl,
or
R¹ and R² are each hydrogen
or
R¹ and R² together with the nitrogen atom to which they are attached form a 4- to 7-membered heterocycle which may contain a further heteroatom from the group consisting of N, O and S,
   where the 4- to 7-membered heterocycle may be substituted by 1 to 2 substituents independently of one another selected from the group consisting of fluorine, trifluoromethyl, (C₁-C₄)-alkyl, trifluoromethyl and (C₁-C₄)-alkoxy,
R³ represents hydrogen or a group of the formula

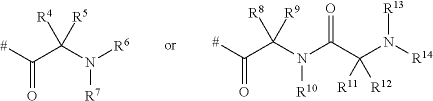

where
# represents the point of attachment to the oxygen atom,
R⁴ represents hydrogen or the side group of a natural α-amino acid or its homologues or isomers,
R⁵ represents hydrogen,
R⁶ represents hydrogen,
R⁷ represents hydrogen,
R⁸ represents hydrogen or the side group of a natural α-amino acid or its homologues or isomers,
R⁹ represents hydrogen,
R¹⁰ represents hydrogen or methyl,
R¹¹ represents hydrogen or the side group of a natural α-amino acid or its homologues or isomers,
R¹² represents hydrogen,
R¹³ represents hydrogen,
and the N-oxides, salts, solvates, salts of the N-oxides and solvates of the N-oxides or salts thereof
in a method for the treatment and/or prevention of acute and/or chronic kidney disorders with or without concomittant acute and/or chronic heart disorders.

Compounds of the invention are the compounds of the formula (I) and the N-oxides, salts, solvates, salts of the N-oxides and solvates of the N-oxides or salts thereof, the compounds that are encompassed by formula (I) and are of the formulae mentioned below and the salts, solvates and solvates of the salts thereof and the compounds that are encompassed by the formula (I) and are mentioned below as embodiments and the salts, solvates and solvates of the salts thereof if the compounds that are encompassed by the formula (I) and are mentioned below are not already salts, solvates and solvates of the salts.

Depending on their structure, the compounds according to the invention may exist in stereoisomeric forms (enantiomers, diastereomers). The invention therefore encompasses the enantiomers or diastereomers and the respective mixtures thereof. It is possible to isolate the stereoisomerically homogeneous constituents from such mixtures of enantiomers and/or diastereomers in a known manner.

If the compounds according to the invention can occur in tautomeric forms, the present invention encompasses all the tautomeric forms.

Preferred salts in the context of the present invention are physiologically acceptable salts of the compounds according to the invention. Also encompassed are salts which are not themselves suitable for pharmaceutical applications but can be used, for example, for isolation or purification of the compounds of the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of conventional bases, by way of example and with preference alkali metal salts (e g sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Designated as solvates in the context of the invention are those forms of the compounds according to the invention which form a complex in the solid or liquid state by coordination with solvent molecules. Hydrates are a specific form of the solvates in which the coordination is with water. Solvates preferred in the context of the present invention are hydrates.

The present invention additionally also encompasses prodrugs of the compounds of the invention. The term “prodrugs” encompasses compounds which for their part may be biologically active or inactive but are converted during their residence time in the body into compounds according to the invention (for example by metabolism or hydrolysis).

In the context of the present invention, unless specified otherwise, the substituents are defined as follows:

Alkyl in the context of the invention is a linear or branched alkyl radical having 1 to 6 or 1 to 4 carbon atoms. Preference is given to a straight-chain or branched alkyl radical having 1 to 4 carbon atoms. Preferred examples include: methyl, ethyl, n-propyl, isopropyl, n-butyl, isobutyl, sec-butyl, tert-butyl, 1-ethylpropyl, n-pentyl and n-hexyl.

Alkenyl in the context of the invention is a straight-chain or branched alkenyl radical having 2 to 4 carbon atoms and a double bond. Preferred examples include: vinyl, allyl, isopropenyl and n-but-2-en-1-yl.

Alkynyl in the context of the invention is a straight-chain or branched alkynyl radical having 2 to 4 carbon atoms and one triple bond. Preferred examples include: ethynyl, n-prop-1-yn-1-yl, n-prop-2-yn-1-yl, n-but-2-yn-1-yl and n-but-3-yn-1-yl.

Alkanediyl in the context of the invention is a linear or branched divalent alkyl radical having 2 to 6 carbon atoms. Preferred examples include: methylene, ethane-1,1-diyl, ethane-1,2-diyl, propane-1,1-diyl, propane-1,2-diyl, propane-2,2-diyl, propane-1,3-diyl, butane-1,4-diyl, butane-1,2-diyl, butane-1,3-diyl, butane-2,3-diyl or butane-3,4-diyl.

Cycloalkyl in the context of the invention is a monocyclic saturated carbocycle having 3 to 7 or 5 to 6 ring carbon atoms. Preferred examples include: cyclopropyl, cyclobutyl, cyclopentyl, cyclohexyl and cycloheptyl.

Alkoxy in the context of the invention is a straight-chain or branched alkoxy radical having 1 to 6, 1 to 4 or 2 to 4 carbon atoms. Preference is given to a straight-chain or branched alkoxy radical having 1 to 4 or 2 to 4 carbon atoms. Preferred examples include: methoxy, ethoxy, n-propoxy, isopropoxy, n-butoxy, tert-butoxy, n-pentoxy and n-hexoxy.

Cycloalkoxy in the context of the invention is a monocyclic saturated carbocycle which has 3 to 7 carbon atoms and is bonded via an oxygen atom. Preferred examples include: cyclopropyloxy, cyclobutyloxy, cyclopentyloxy, cyclohexyloxy and cycloheptylov.

Alkylsulphanyl in the context of the invention is a straight-chain or branched alkyl radical which has 1 to 4 carbon atoms and is attached via a sulphanyl group. Preferred examples include: methylsulphanyl, ethylsulphanyl, n-propylsulphanyl, isopropylsulphanyl, n-butylsulphanyl and tert-butylsulphanyl.

Alkylsulphonyl in the context of the invention is a straight-chain or branched alkyl radical which has 1 to 4 carbon atoms and is attached via a sulphonyl group. Preferred examples include: methylsulphonyl, ethylsulphonyl, n-propylsulphonyl, isopropylsulphonyl, n-butylsulphonyl and tert-butylsulphonyl.

Heterocycle in the context of the invention is a saturated heterocycle which has a total of 4 to 7 ring atoms, contains one or two ring heteroatoms from the group consisting of N, O and S and is attached via a ring carbon atom or optionally a ring nitrogen atom. Examples include: azetidinyl, pyrrolidinyl, pyrazolidinyl, tetrahydrofumnyl, piperidinyl, piperazinyl, tetmhydropyranyl, morpholinyl, thiomorpholinyl and azepanyl. Preference is given to azetidinyl, pyrrolidinyl, tetmhydrofuranyl, piperidinyl, piperazinyl, tetmhydropyranyl and morpholinyl. Particular preference is given to azetidinyl, pyrrolidinyl, piperidinyl and morpholinyl.

The side group of an α-amino acid in the meaning of R³ embraces both the side groups of the naturally α-amino acids occurring and the side groups of homologues and isomers of these α-amino acids. Here, the α-amino acid may be present either in the L- or in the D-configuration or else as a mixture of the L- and D-Form. Examples of side groups include: methyl (alanine), propan-2-yl (valine), propan-1-yl (norvaline), 2-methylpropan-1-yl (leucine), 1-methylpropan-1-yl (isoleucine), butan-1-yl (norleucine), tert-butyl (2-tert-butylglycine), phenyl (2-phenylglycine), benzyl (phenylalanine), p-hydroxybenzyl (tyrosine), indol-3-ylmethyl (tryptophan), imidazol-4-ylmethyl (histidine), hydroxymethyl (serine), 2-hydroxyethyl (homoserine), 1-hydroxyethyl (threonine), mercaptomethyl (cysteine), methylthiomethyl (S-methylcysteine), 2-mercaptoethyl (homocysteine), 2-methylthioethyl (methionine), carbamoylmethyl (asparagine), 2-carbamoylethyl (glutamine), carboxymethyl (aspartic acid), 2-carboxyethyl (glutamic acid), 4-aminobutan-1-yl (lysine), 4-amino-3-hydroxybutan-1-yl (hydroxylysine), 3-aminopropan-1-yl (ornithine), 2-aminoethyl (2,4-diaminobutyric acid), aminomethyl (2,3-diaminopropionic acid), 3-guanidinopropan-1-yl (arginine), 3-ureidopropan-1-yl (citrulline). Preferred α-amino acid side groups in the meaning of R³ are methyl (alanine), propan-2-yl (valine), 2-methylpropan-1-yl (leucine), benzyl (phenylalanine), imidazol-4-ylmethyl (histidine), hydroxymethyl (serine), 1-hydroxyethyl (threonine), 4-aminobutan-1-yl (lysine), 3-aminopropan-1-yl (ornithine), 2-aminoethyl (2,4-diaminobutyric acid), aminomethyl (2,3-diaminopropionic acid), 3-guanidinopropan-1-yl (arginine). In each case, preference is given to the Lconfiguration.

An oxo group in the context of the invention is an oxygen atom attached to a carbon atom via a double bond.

When radicals in the compounds of the invention are substituted, the radicals may be mono- or polysubstituted, unless specified otherwise. In the context of the present invention, all radicals which occur more than once are defined independently of one another. Substitution by one, two or three identical or different substituents is preferred. Substitution by one or two identical or different substituents is very particularly preferred.

In the context of the present invention, preference is given to the use of compounds of the formula (I) in which
R¹ represents hydrogen or (C₁-C₃)-alkyl,
R² represents hydrogen or (C₁-C₃)-alkyl,
   where (C₁-C₃)-alkyl may be substituted by 1 or 2 substituents independently of one another selected from the group consisting of fluorine, trifluoromethyl, methoxy, ethoxy, cyclopropyl and cyclobutyl,
or
R¹ and R² together with the nitrogen atom to which they are attached form a 4- to 6-membered heterocycle which may contain a further heteroatom from the group consisting of N, O and S,
   where the 4- to 6-membered heterocycle may be substituted by 1 to 2 substituents independently of one another selected from the group consisting of fluorine, trifluoromethoxy, (C₁-C₄)-alkyl, trifluoromethoxy, methoxy and ethoxy,
R³ represents hydrogen or a group of the formula

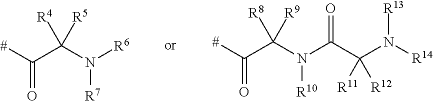

where
# represents the point of attachment to the oxygen atom,
R⁴ represents 3-aminopropan-1-yl,
R⁵ represents hydrogen,
R⁶ represents hydrogen,
R⁷ represents hydrogen,
R⁸ represents methyl,
R⁹ represents hydrogen,
R¹⁰ represents hydrogen,
R¹¹ represents methyl, 2-aminoeth-1-yl, 4-aminobut-1-yl, 3-guanidinopropan-1-yl or imidazol-4-ylmethyl,
R¹² represents hydrogen,
R¹³ represents hydrogen,
R¹⁴ represents hydrogen,
and the N-oxides, salts, solvates, salts of the N-oxides and solvates of the N-oxides or salts thereof.

In the context of the present invention, preference is furthermore given to the use of compounds of the formula (I) in which
R¹ represents (C₁-C₃)-alkyl,
R² represents (C₁-C₃)-alkyl,
   where (C₁-C₃)-alkyl may be substituted by 1 or 2 substituents independently of one another selected from the group consisting of fluorine, trifluoromethyl, methoxy, ethoxy, cyclopropyl and cyclobutyl,
or
R¹ and R² each represent hydrogen
or
R¹ and R² together with the nitrogen atom to which they are attached form a 4- to 6-membered heterocycle which may contain a further heteroatom from the group consisting of N, O and S,
   where the 4- to 6-membered heterocycle may be substituted by 1 or 2 substituents independently of one another selected from the group consisting of fluorine, trifluoromethoxyl, (C₁-C₄)-alkyl, trifluoromethoxy, methoxy and ethoxy,
R³ represents hydrogen or a group of the formula

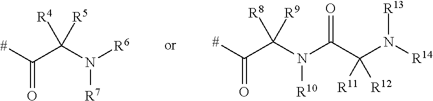

where
# represents the point of attachment to the oxygen atom,
R⁴ represents 3-aminopropan-1-yl,
R⁵ represents hydrogen,
R⁶ represents hydrogen,
R⁷ represents hydrogen,
R⁸ represents methyl,
R⁹ represents hydrogen,
R¹⁰ represents hydrogen,
R¹¹ represents methyl, 2-aminoeth-1-yl, 4-aminobut-1-yl, 3-guanidinopropan-1-yl or imidazol-4-ylmethyl,
R¹² represents hydrogen,
R¹³ represents hydrogen,
R¹⁴ represents hydrogen,
and the N-oxides, salts, solvates, salts of the N-oxides and solvates of the N-oxides or salts thereof.

In the context of the present invention, particular preference is given to the use of compounds of the formula (I) in which
R¹ represents hydrogen or ethyl,
R² represents hydrogen or ethyl,
or
R¹ and R² together with the nitrogen atom to which they are attached form a 4- to 6-membered heterocycle which may contain a further heteroatom from the group consisting of N, O and S,
R³ represents hydrogen or a group of the formula

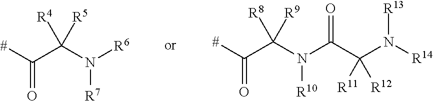

where
# represents the point of attachment to the oxygen atom,
R⁴ represents 3-aminopropan-1-yl,
R⁵ represents hydrogen,
R⁶ represents hydrogen,
R⁷ represents hydrogen,
R⁸ represents methyl,
R⁹ represents hydrogen,
R¹⁰ represents hydrogen,
R¹¹ represents methyl, 2-aminoeth-1-yl, 4-aminobut-1-yl, 3-guanidinopropan-1-yl or imidazol-4-ylmethyl,
R¹² represents hydrogen,
R¹³ represents hydrogen,
R¹⁴ represents hydrogen,
and the N-oxides, salts, solvates, salts of the N-oxides and solvates of the N-oxides or salts thereof.

In the context of the present invention, preference is furthermore given to the use of compounds of the formula (I) in which
R¹ represents ethyl,
R² represents ethyl,
or
R¹ and R² are each hydrogen
or
R¹ and R² together with the nitrogen atom to which they are attached form a 4- to 6-membered heterocycle which may contain a further heteroatom from the group consisting of N, O and S,
R³ represents hydrogen or a group of the formula

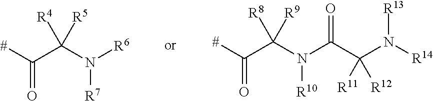

where
# represents the point of attachment to the oxygen atom,
R⁴ represents 3-aminopropan-1-yl,
R⁵ represents hydrogen,
R⁶ represents hydrogen,
R⁷ represents hydrogen,
R⁸ represents methyl,
R⁹ represents hydrogen,
R¹⁰ represents hydrogen,
R¹¹ represents methyl, 2-aminoeth-1-yl, 4-aminobut-1-yl, 3-guanidinopropan-1-yl or imidazol-4-ylmethyl,
R¹² represents hydrogen,
R¹³ represents hydrogen,
R¹⁴ represents hydrogen,
and the N-oxides, salts, solvates, salts of the N-oxides and solvates of the N-oxides or salts thereof.

In the context of the present invention, preference is furthermore given to the use of compounds of the formula (I) in which
R¹ represents hydrogen or ethyl,
R² represents hydrogen or ethyl,
or
R¹ and R² together with the nitrogen atom to which they are attached form an azetidinyl, pyrrolidinyl or piperidinyl ring,
where the azetidinyl ring may be substituted by a methoxy substituent,
R³ represents hydrogen or a group of the formula

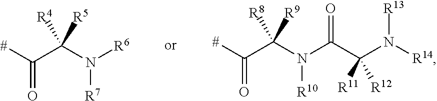

where
# represents the point of attachment to the oxygen atom,
R⁴ represents 3-aminopropan-1-yl,
R⁵ represents hydrogen,
R⁶ represents hydrogen,
R⁷ represents hydrogen,
R⁸ represents methyl,
R⁹ represents hydrogen,
R¹⁰ represents hydrogen,
R¹¹ represents methyl, 2-aminoeth-1-yl, 4-aminobut-1-yl, 3-guanidinopropan-1-yl or imidazol-4-ylmethyl,
R¹² represents hydrogen,
R¹³ represents hydrogen,
R¹⁴ represents hydrogen,
and the N-oxides, salts, solvates, salts of the N-oxides and solvates of the N-oxides or salts thereof.

In the context of the present invention, preference is furthermore given to the use of compounds of the formula (I) in which
R¹ represents ethyl,
R² represents ethyl,
or
R¹ and R² represent hydrogen
or
R¹ and R² together with the nitrogen atom to which they are attached form an azetidinyl, pyrrolidinyl or piperidinyl ring,
where the azetidinyl ring may be substituted by a methoxy substituent,
R³ represents hydrogen or a group of the formula

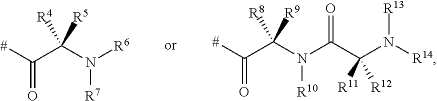

where
# represents the point of attachment to the oxygen atom,
R⁴ represents 3-aminopropan-1-yl,
R⁵ represents hydrogen,
R⁶ represents hydrogen,
R⁷ represents hydrogen,
R⁸ represents methyl,
R⁹ represents hydrogen,
R¹⁰ represents hydrogen,
R¹¹ represents methyl, 2-aminoeth-1-yl, 4-aminobut-1-yl, 3-guanidinopropan-1-yl or imidazol-4-ylmethyl,
R¹² represents hydrogen,
R¹³ represents hydrogen,
R¹⁴ represents hydrogen,
and the N-oxides, salts, solvates, salts of the N-oxides and solvates of the N-oxides or salts thereof.

In the context of the present invention, preference is furthermore given to the use of compounds of the formula (I) in which
R¹ represents hydrogen,
R² represents hydrogen,
or
R¹ and R² together with the nitrogen atom to which they are attached form a pyrrolidinyl ring,
R³ represents a group of the formula

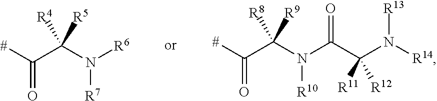

where
# represents the point of attachment to the oxygen atom,
R⁴ represents 3-aminopropan-1-yl,
R⁵ represents hydrogen,
R⁶ represents hydrogen,
R⁷ represents hydrogen,
R⁸ represents methyl,
R⁹ represents hydrogen,
R¹⁰ represents hydrogen,
R¹¹ represents methyl, 2-aminoeth-1-yl, 4-aminobut-1-yl, 3-guanidinopropan-1-yl or imidazol-4-ylmethyl,
R¹² represents hydrogen,
R¹³ represents hydrogen,
R¹⁴ represents hydrogen,
and the N-oxides, salts, solvates, salts of the N-oxides and solvates of the N-oxides or salts thereof.

In the context of the present invention, preference is furthermore also given to the use of compounds of the formula (I) in which
R¹ represents hydrogen or ethyl,
R² represents hydrogen or ethyl,
or
R¹ and R² together with the nitrogen atom to which they are attached form an azetidinyl, pyrrolidinyl or piperidinyl ring,
   where the azetidinyl ring may be substituted by a methoxy substituent,
R³ represents a group of the formula

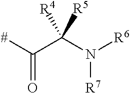

where
# represents the point of attachment to the oxygen atom,
R⁴ represents 3-aminopropan-1-yl,
R⁵ represents hydrogen,
R⁶ represents hydrogen,
R⁷ represents hydrogen,
and the N-oxides, salts, solvates, salts of the N-oxides and solvates of the N-oxides or salts thereof.

In the context of the present invention, preference is furthermore also given to the use of compounds of the formula (I) in which
R¹ represents ethyl,
R² represents ethyl,
or
R¹ and R² represent hydrogen
or
R¹ and R² together with the nitrogen atom to which they are attached form an azetidinyl, pyrrolidinyl or piperidinyl ring,
   where the azetidinyl ring may be substituted by a methoxy substituent,
R³ represents a group of the formula

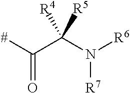

where
# represents the point of attachment to the oxygen atom,
R⁴ represents 3-aminopropan-1-yl,
R⁵ represents hydrogen,
R⁶ represents hydrogen,
R⁷ represents hydrogen,
and the N-oxides, salts, solvates, salts of the N-oxides and solvates of the N-oxides or salts thereof.

In the context of the present invention, particular preference is also given to the use of compounds of the formula (I) in which
R¹ represents hydrogen or ethyl,
R² represents hydrogen or ethyl,
or
R⁷ and R¹ together with the nitrogen atom to which they are attached form an azetidinyl, pyrrolidinyl or piperidinyl ring,
   where the azetidinyl ring may be substituted by a methoxy substituent,
R³ represents a group of the formula

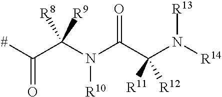

where
# represents the point of attachment to the oxygen atom,
R⁸ represents methyl,
R⁹ represents hydrogen,
R¹⁰ represents hydrogen,
R¹¹ represents methyl, 2-aminoeth-1-yl, 4-aminobut-1-yl, 3-guanidinopropan-1-yl or imidazol-4-ylmethyl,
R¹² represents hydrogen,
R¹³ represents hydrogen,
R¹⁴ represents hydrogen,
and the N-oxides, salts, solvates, salts of the N-oxides and solvates of the N-oxides or salts thereof.

In the context of the present invention, particular preference is also given to the use of compounds of the formula (I) in which
R¹ represents ethyl,
R² represents ethyl,
or
R¹ and R² represent hydrogen
or
R¹ and R² together with the nitrogen atom to which they are attached form an azetidinyl, pyrrolidinyl or piperidinyl ring,
   where the azetidinyl ring may be substituted by a methoxy substituent,
R³ represents a group of the formula

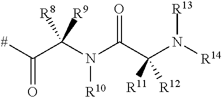

where
# represents the point of attachment to the oxygen atom,
R⁸ represents methyl,
R⁹ represents hydrogen,
R¹⁰ represents hydrogen,
R¹¹ represents methyl, 2-aminoeth-1-yl, 4-aminobut-1-yl, 3-guanidinopropan-1-yl or imidazol-4-ylmethyl,
R¹² represents hydrogen,
R¹³ represents hydrogen,
R¹⁴ represents hydrogen,
and the N-oxides, salts, solvates, salts of the N-oxides and solvates of the N-oxides or salts thereof.

In the context of the present invention, particular preference is also given to the use of compounds of the formula (I) in which
R¹ represents hydrogen or ethyl,
R² represents hydrogen or ethyl,
or
R¹ and R² together with the nitrogen atom to which they are attached form an azetidinyl, pyrrolidinyl or piperidinyl ring,
   where the azetidinyl ring may be substituted by a methoxy substituent,
R³ represents hydrogen,
and the N-oxides, salts, solvates, salts of the N-oxides and solvates of the N-oxides or salts thereof.

In the context of the present invention, particular preference is also given to the use of compounds of the formula (I) in which
R¹ represents ethyl,
R² represents ethyl,
or
R¹ and R² represent hydrogen
or
R¹ and R² together with the nitrogen atom to which they are attached form an azetidinyl, pyrrolidinyl or piperidinyl ring,
   where the azetidinyl ring may be substituted by a methoxy substituent,
R³ represents hydrogen,
and the N-oxides, salts, solvates, salts of the N-oxides and solvates of the N-oxides or salts thereof.

In the context of the present invention, preference is also given to the use of compounds of the formula (I) in which R³ represents hydrogen, and the N-oxides, salts, solvates, salts of the N-oxides and solvates of the N-oxides or salts thereof.

In the context of the present invention, preference is also given to the use of compounds of the formula (I) in which
R³ represents hydrogen or a group of the formula

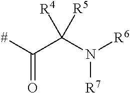

where
# represents the point of attachment to the oxygen atom,
R⁴ represents 3-aminopropan-1-yl,
R⁵ represents hydrogen,
R⁶ represents hydrogen,
R⁷ represents hydrogen,
and the N-oxides, salts, solvates, salts of the N-oxides and solvates of the N-oxides or salts thereof.

In the context of the present invention, preference is also given to the use of compounds of the formula (I) in which
R³ represents hydrogen or a group of the formula

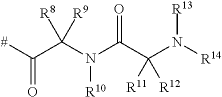

where
# represents the point of attachment to the oxygen atom,
R⁸ represents methyl,
R⁹ represents hydrogen,
R¹⁰ represents hydrogen,
R¹¹ represents methyl, 2-aminoeth-1-yl, 4-aminobut-1-yl, 3-guanidinopropan-1-yl or imidazol-4-ylmethyl,
R¹² represents hydrogen,
R¹³ represents hydrogen or methyl,
R¹⁴ represents hydrogen or methyl,
and the N-oxides, salts, solvates, salts of the N-oxides and solvates of the N-oxides or salts thereof.

In the context of the present invention, preference is also given to the use of compounds of the formula (I) in which
R¹ represents hydrogen or (C₁-C₃)-alkyl,
R² represents hydrogen or (C₁-C₃)-alkyl,
   where (C₁-C₃)-alkyl may be substituted by 1 or 2 substituents independently of one another selected from the group consisting of fluorine, trifluoromethyl, methoxy, ethoxy, cyclopropyl and cyclobutyl,
or
R¹ and R² together with the nitrogen atom to which they are attached form a 4- to 6-membered heterocycle which may contain a further heteroatom from the group consisting of N, O and S,
   where the 4- to 6-membered heterocycle may be substituted by 1 or 2 substituents independently of one another selected from the group consisting of fluorine, trifluoromethyl, methyl, ethyl, methoxy and ethoxy,
and the N-oxides, salts, solvates, salts of the N-oxides and solvates of the N-oxides or salts thereof.

In the context of the present invention, preference is also given to the use of compounds of the formula (I) in which
R¹ represents (C₁-C₃)-alkyl,
R² represents (C₁-C₃)-alkyl,
   where (C₁-C₃)-alkyl may be substituted by 1 or 2 substituents independently of one another selected from the group consisting of fluorine, trifluoromethyl, methoxy, ethoxy, cyclopropyl and cyclobutyl,
or
R¹ and R² represent hydrogen
or
R¹ and R² together with the nitrogen atom to which they are attached form a 4- to 6-membered heterocycle which may contain a further heteroatom from the group consisting of N, O and S,
   where the 4- to 6-membered heterocycle may be substituted by 1 or 2 substituents independently of one another selected from the group consisting of fluorine, trifluoromethyl, methyl, ethyl, methoxy and ethoxy,
and the N-oxides, salts, solvates, salts of the N-oxides and solvates of the N-oxides or salts thereof.

In the context of the present invention, preference is also given to the use of compounds of the formula (I) in which
R³ represents a group of the formula

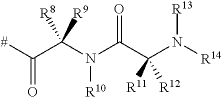

where
# represents the point of attachment to the oxygen atom,
R⁸ represents methyl,
R⁹ represents hydrogen,
R¹⁰ represents hydrogen,
R¹¹ represents methyl, 2-aminoeth-1-yl, 4-aminobut-1-yl, 3-guanidinopropan-1-yl or imidazol-4-ylmethyl,
R¹² represents hydrogen,
R¹³ represents hydrogen,
R¹⁴ represents hydrogen,
and the N-oxides, salts, solvates, salts of the N-oxides and solvates of the N-oxides or salts thereof.

In the context of the present invention, preference is also given to the use of compounds of the formula (I) in which
R¹ represents hydrogen,
R² represents hydrogen,
or
R¹ and R² together with the nitrogen atom to which they are attached form an azetidinyl, pyrrolidinyl or piperidinyl ring,
   where the azetidinyl, pyrrolidinyl or piperidinyl ring may be substituted by 1 or 2 substituents independently of one another selected from the group consisting of fluorine, trifluoromethyl, methyl, ethyl, methoxy and ethoxy,
and the N-oxides, salts, solvates, salts of the N-oxides and solvates of the N-oxides or salts thereof.

In the context of the present invention, preference is also given to the use of compounds of the formula (I) in which
R¹ represents hydrogen,
R² represents hydrogen,
or
R¹ and R² together with the nitrogen atom to which they are attached form an azetidinyl, pyrrolidinyl or piperidinyl ring,
and the N-oxides, salts, solvates, salts of the N-oxides and solvates of the N-oxides or salts thereof.

In the context of the present invention, preference is also given to the use of compounds of the formula (I) in which
R¹ represents hydrogen,
R² represents hydrogen,
or
R¹ and R² together with the nitrogen atom to which they are attached form a pyrrolidinyl ring,
and the N-oxides, salts, solvates, salts of the N-oxides and solvates of the N-oxides or salts thereof.

In the context of the present invention, particular preference is given to the use of the following compounds:
2-amino-6-({[2-(4-chlorophenyl)-1,3-thiazol-4-yl]methyl}sulphanyl)-4-[4-(2-hydroxyethoxy)phenyl]pyridine-3,5-dicarbonitrile
2-({[2-(4-chlorophenyl)-1,3-thiazol-4-yl]methyl}sulphanyl)-6-(diethylamino)-4-[4-(2-hydroxyethoxy)phenyl]pyridine-3,5-dicarbonitrile
2-({[2-(4-chlorophenyl)-1,3-thiazol-4-yl]methyl}sulphanyl)-4-[4-(2-hydroxyethoxy)phenyl]-6-(3-methoxyazetidin-1-yl)pyridine-3,5-dicarbonitrile
2-({[2-(4-chlorophenyl)-1,3-thiazol-4-yl]methyl}sulphanyl)-4-[4-(2-hydroxyethoxy)phenyl]-6-(pyrrolidin-1-yl)pyridine-3,5-dicarbonitrile
2-({[2-(4-chlorophenyl)-1,3-thiazol-4-yl]methyl}sulphanyl)-4-[4-(2-hydroxyethoxy)phenyl]-6-(piperidin-1-yl)pyridine-3,5-dicarbonitrile,
and the N-oxides, salts, solvates, salts of the N-oxides and solvates of the N-oxides or salts thereof.

In the context of the present invention, particular preference is furthermore given to the use of the following compounds:
2-({[2-(4-chlorophenyl)-1,3-thiazol-4-yl]methyl}sulphanyl)-6-(diethylamino)-4-[4-(2-hydroxyethoxy)phenyl]pyridine-3,5-dicarbonitrile
2-({[2-(4-chlorophenyl)-1,3-thiazol-4-yl]methyl}sulphanyl)-4-[4-(2-hydroxyethoxy)phenyl]-6-(3-methoxyazetidin-1-yl)pyridine-3,5-dicarbonitrile
2-({[2-(4-chlorophenyl)-1,3-thiazol-4-yl]methyl}sulphanyl)-4-[4-(2-hydroxyethoxy)phenyl]-6-(pyrrolidin-1-yl)pyridine-3,5-dicarbonitrile
2-({[2-(4-chlorophenyl)-1,3-thiazol-4-yl]methyl}sulphanyl)-4-[4-(2-hydroxyethoxy)phenyl]-6-(piperidin-1-yl)pyridine-3,5-dicarbonitrile,
and the N-oxides, salts, solvates, salts of the N-oxides and solvates of the N-oxides or salts thereof.

In the context of the present invention, particular preference is furthermore given to the use of the following compounds:
2-{4-[2-({[2-(4-chlorophenyl)-1,3-thiazol-4-yl]methyl}sulfanyl)-3,5-dicyano-6-(pyrrolidin-1-yl)pyridin-4-yl]phenoxy}ethyl L-ornithinate bis(trifluoroacetate) or
2-{4-[2-amino-6-({[2-(4-chlorophenyl)-1,3-thiazol-4-yl]methyl}sulphanyl)-3,5-dicyanopyridin-4-yl]phenoxy}ethyl L-ornithinate dihydrochloride,
and the N-oxides, salts, solvates, salts of the N-oxides and solvates of the N-oxides or salts thereof.

In the context of the present invention, special preference is given to the use of the following compounds:
2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-N-[(2 S)-2,4-diaminobutanoyl] L-alaninate dihydrochloride
2-{4-[2-amino-6-({[2-(4-chlorophenyl)-1,3-thiazol-4-yl]methyl}sulphanyl)-3,5-dicyanopyridin-4-yl]phenoxy}ethyl-N-[(2S)-2,4-diaminobutanoyl] L-alaninate dihydrochloride
2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-L-lysyl L-alaninate dihydrochloride
2-{4-[2-amino-6-({[2-(4-chlorophenyl)-1,3-thiazol-4-yl]methyl}sulphanyl)-3,5-dicyanopyridin-4-yl]phenoxy}ethyl-L-lysyl L-alaninate dihydrochloride
2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-L-alanyl L-alaninate hydrochloride
2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-L-argyl L-alaninate dihydrochloride
2-{4-[2-amino-6-({[2-(4-chlorophenyl)-1,3-thiazol-4-yl]methyl}sulphanyl)-3,5-dicyanopyridin-4-yl]phenoxy}ethyl-L-argyl L-alaninate dihydrochloride
2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-L-histidyl L-alaninate dihydrochloride
2-{4-[2-amino-6-({[2-(4-chlorophenyl)-1,3-thiazol-4-yl]methyl}sulphanyl)-3,5-dicyanopyridin-4-yl]phenoxy}ethyl-L-histidyl L-alaninate dihydrochlorid,
and the N-oxides, salts, solvates, salts of the N-oxides and solvates of the N-oxides or salts thereof.

In the context of the present invention, special preference is given to the use of the following compounds:
2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-N-[(2S)-2,4-diaminobutanoyl] L-alaninate dihydrochloride
2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-L-lysyl L-alaninate dihydrochloride
2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-L-alanyl L-alaninate hydrochloride
2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-L-argyl L-alaninate dihydrochloride
2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-L-histidyl L-alaninate dihydrochloride
and the N-oxides, salts, solvates, salts of the N-oxides and solvates of the N-oxides or salts thereof.

In the context of the present invention, special preference is given to the use of the following compounds:
2-{4-[2-amino-6-({[2-(4-chlorophenyl)-1,3-thiazol-4-yl]methyl}sulphanyl)-3,5-dicyanopyridin-4-yl]phenoxy}ethyl-N-[(2S)-2,4-diaminobutanoyl] L-alaninate dihydrochloride
2-{4-[2-amino-6-({[2-(4-chlorophenyl)-1,3-thiazol-4-yl]methyl}sulphanyl)-3,5-dicyanopyridin-4-yl]phenoxy}ethyl-L-lysyl L-alaninate dihydrochloride
2-{4-[2-amino-6-({[2-(4-chlorophenyl)-1,3-thiazol-4-yl]methyl}sulphanyl)-3,5-dicyanopyridin-4-yl]phenoxy}ethyl-L-argyl L-alaninate dihydrochloride
2-{4-[2-amino-6-({[2-(4-chlorophenyl)-1,3-thiazol-4-yl]methyl}sulphanyl)-3,5-dicyanopyridin-4-yl]phenoxy}ethyl-L-histidyl L-alaninate dihydrochlorid,
and the N-oxides, salts, solvates, salts of the N-oxides and solvates of the N-oxides or salts thereof.

In the context of the present invention, special preference is given to the use of the following compound:
2-amino-6-({[2-(4-chlorophenyl)-1,3-thiazol-4-yl]methyl}sulphanyl)-4-[4-(2-hydroxyethoxy)phenyl] pyridine-3,5-dicarbonitrile
and the N-oxides, salts, solvates, salts of the N-oxides and solvates of the N-oxides or salts thereof.

In the context of the present invention, special preference is furthermore given to the use of the following compound:
2-({[2-(4-chlorophenyl)-1,3-thiazol-4-yl]methyl}sulphanyl)-4-[4-(2-hydroxyethoxy)phenyl]-6-(pyrrolidin-1-yl)pyridine-3,5-dicarbonitrile
and the N-oxides, salts, solvates, salts of the N-oxides and solvates of the N-oxides or salts thereof.

In the context of the present invention, special preference is furthermore given to the use of the following compound:
2-{4-[2-({[2-(4-chlorophenyl)-1,3-thiazol-4-yl]methyl}sulphanyl)-3,5-dicyano-6-(pyrrolidin-1-yl)pyridin-4-yl]phenoxy}ethyl-L-alanyl L-alaninate hydrochloride
and the N-oxides, salts, solvates, salts of the N-oxides and solvates of the N-oxides or salts thereof.

In the context of the present invention, special preference is furthermore given to the use of the following compound:
2-{4-[2-amino-6-({[2-(4-chlorophenyl)-1,3-thiazol-4-yl]methyl}sulphanyl)-3,5-dicyanopyridin-4-yl]phenoxy}ethyl-L-lysyl L-alaninate dihydrochloride
and the N-oxides, salts, solvates, salts of the N-oxides and solvates of the N-oxides or salts thereof.

Surprisingly, the compounds according to the invention exhibit an unforeseeable useful spectrum of pharmacological action and are therefore particularly suitable for the prevention and/or treatment of disorders, in particular of acute and/or chronic kidney disorders.

The compounds according to the invention show an advantageous spectrum of therapeutic and/or pharmacological activity.

“Selective ligands on adenosine A1 receptors” denote, in the context of the present invention, those adenosine receptor ligands for which we observe on the one hand a definite action on the A1 adenosine receptor subtypes and on the other hand no or a markedly weaker action (factor of 10 or higher) on A2a, A2b and A3 adenosine receptor subtypes, referring to the test methods for the selectivity of action (see Methods B-1).

The compounds according to the invention can function as full or as partial adenosine receptor agonists, depending on their respective structure. Partial adenosine receptor agonists are defined herein as receptor ligands that trigger a functional response to adenosine receptors, which is less than with full agonists (for example adenosine itself). Partial agonists consequently have lower efficacy with respect to receptor activation than full agonists.

Partial adenosine A1 agonists can be used in kidney disorders even when associated with other disorders, e.g. of the cardiovascular system.

The partial A1 agonists according to the invention are suitable for preventing or treating acute kidney disorders with or without concomitant acute and/or chronic heart disorders.

The partial A1 agonists according to the invention are suitable for preventing or treating chronic kidney disorders with or without concomitant acute and/or chronic heart disorders.

The present invention further provides for the use of the compounds according to the invention for treatment and/or prevention of disorders, in particular the disorders mentioned above.

The present invention further provides for the use of the partial A1 agonists for producing a medicament for the treatment and/or prevention of disorders, in particular the disorders mentioned above.

The present invention further provides a process for treatment and/or prevention of disorders, in particular the disorders mentioned above, using an effective amount of at least one of the partial A1 agonists.

The present invention further provides the partial A1 agonists for use in a method for treatment and/or prophylaxis of acute kidney disorders.

The present invention further provides the partial A1 agonists for use in a method for treatment and/or prophylaxis of chronic kidney disorders.

The present invention further provides the partial A1 agonists for use in a method for the treatment and/or prophylaxis of acute kidney disorders in combination with coronary heart disease, acute coronary syndrome, angina pectoris, heart failure, myocardial infarction and atrial fibrillation.

The present invention further provides the partial A1 agonists for use in a method for the treatment and/or prophylaxis of chronic kidney disorders in combination with coronary heart disease, acute coronary syndrome, angina pectoris, heart failure, myocardial infarction and atrial fibrillation.

The present invention further provides the partial A1 agonists for use in a method for the treatment and/or prophylaxis of chronic kidney disorders in combination with diabetes, metabolic syndrome and dyslipidaemias.

The partial A1 agonists can be used alone or, if required, in combination with other active compounds. The present invention further provides medicaments comprising at least one of the compounds of the invention and one or more further active compounds, in particular for the treatment and/or prevention of the disorders mentioned above.

Active compounds suitable for combination are, by way of example and with preference: active compounds which modulate lipid metabolism, antidiabetics, hypotensive agents, perfusion-enhancing and/or antithrombotic agents, antioxidants, chemokine receptor antagonists, p38 kinase inhibitors, NPY agonists, orexin agonists, anorectics, PAF-AH inhibitors, antiphlogistics (COX inhibitors, LTB4-receptor antagonists), analgesics, for example aspirin, antidepressants and other psychopharmaceuticals.

The present invention provides in particular combinations of at least one of the partial A1 agonists and at least one lipid metabolism-modifying active compound, antidiabetic, hypotensive active compound and/or agent having antithrombotic action.
lipid metabolism-modulating active compounds, by way of example and with preference from the group of the HMG-CoA reductase inhibitors, inhibitors of HMG-CoA reductase expression, squalene synthesis inhibitors, ACAT inhibitors, LDL receptor inductors, cholesterol absorption inhibitors, polymeric bile acid adsorbs, bile acid reabsorption inhibitors, MTP inhibitors, lipase inhibitors, LpL activators, fibrates, niacin, CETP inhibitors, PPAR-α, PPAR-γ and/or PPAR-δ agonists, RXR modulators, FXR modulators, LXR modulators, thyroid hormones and/or thyroid mimetics, ATP citrate lyase inhibitors, Lp(a) antagonists, cannabinoid receptor 1 antagonists, leptin receptor agonists, bombesin receptor agonists, histamine receptor agonists and the antioxidants/radical scavengers;
antidiabetics mentioned in the Rote Liste 2004/II, chapter 12, and also, by way of example and with preference, those from the group of the sulphonylureas, biguanides, meglitinide derivatives, glucosidase inhibitors, inhibitors of dipeptidyl-peptidase IV (DPP-IV inhibitors), oxadiazolidinones, thiazolidinediones, GLP 1 receptor agonists, glucagon antagonists, insulin sensitizers, CCK 1 receptor agonists, leptin receptor agonists, inhibitors of liver enzymes involved in the stimulation of gluconeogenesis and/or glycogenolysis, modulators of glucose uptake and also potassium channel openers, such as, for example, those disclosed in WO 97/26265 and WO 99/03861,
hypotensive active compounds, by way of example and with preference from the group of the calcium antagonists, angiotensin II ATI receptor antagonists, ACE inhibitors, renin inhibitors, beta-receptor blockers, alpha-receptor blockers, aldosterone antagonists, mineralocorticoid receptor antagonists, ECE inhibitors, ACE/NEP inhibitors and the vasopeptidase inhibitors,
agents with antithrombotic action, by way of example and with preference from the group of the platelet aggregation inhibitors, the anticoagulants and the profibrinolytic substances, factor Xa inhibitors and vitamin K antagonists,
agents with anti-inflammatory action, by way of example and with preference from the group of glucocorticosteroids, nonsteroidal antiphlogistics or nonsteroidal antirheumatics,
diuretics, by way of example and with preference loop diuretics, thiazides, potassium-sparing diuretics, or carbonic anhydrase inhibitors,
organic nitrates and NO donors, for example sodium nitroprusside, nitroglycerin, isosorbide mononitrate, isosorbide dinitrate, molsidomine or SIN-1, and inhaled NO;
compounds which inhibit the degradation of cyclic guanosine monophosphate (cGMP) and/or cyclic adenosine monophosphate (cAMP), for example inhibitors of phosphodiesterases (PDE) 1, 2, 3, 4 and/or 5, such as, by way of example, sildenafil, vardenafil, tadalafil and milrinone;
agents with positive inotropic action, for example digitoxin, digoxin, epinephrine, norepinephrine, dobutamine and dopamine,
agents with antiproliferative action, for example multikinase inhibitors and preferably sorafenib, imatinib, gefitinib and erlotinib
compounds with positive inotropic activity;
natriuretic peptides, for example atrial natriuretic peptide (ANP, anaritide), B-type natriuretic peptide or brain natriuretic peptide (BNP, nesiritide), C-type natriuretic peptide (CNP) and urodilatin;
agonists of the prostacyclin receptor (IP receptor), for example iloprost, beraprost, cicaprost;
agonists of the relaxin receptor-1 (RXFP-1), for example serelaxin
inhibitors of the If (funny channel) channel, for example ivabradine;
calcium sensitizers, by way of example and with preference levosimendan;
potassium supplements;
NO-independent but haem-dependent stimulators of guanylate cyclase, such as especially the compounds described in WO 00/06568, WO 00/06569, WO 02/42301 and WO 03/095451;
NO- and haem-independent activators of guanylate cyclase, such as especially the compounds described in WO 01/19355, WO 01/19776, WO 01/19778, WO 01/19780, WO 02/070462 and WO 02/070510;
inhibitors of human neutrophil elastase (HNE), for example sivelestat and DX-890 (Reltran);
compounds which inhibit the signal transduction cascade, for example tyrosine kinase inhibitors, especially sorafenib, imatinib, gefitinib and erlotinib; and/or
compounds which influence the energy metabolism of the heart, for example etomoxir, dichloroacetate, ranolazine and trimetazidine.

In a preferred embodiment of the invention, the compounds of the invention are administered in combination with a CETP inhibitor, by way of example and with preference dalcetrapib, BAY 60-5521, anacetrapib, torcetrapib, JTT-705 or CETP vaccine (CETi-1).

In a preferred embodiment of the invention, the compounds of the invention are administered in combination with a thyroid receptor agonist, by way of example and with preference D-thyroxine, 3,5,3′-triiodothyronine (T3), CGS 23425 or axitirome (CGS 26214).

In a preferred embodiment of the invention, the compounds of the invention are administered in combination with an HMG-CoA reductase inhibitor from the class of statins, by way of example and with preference lovastatin, cerivastatin, simvastatin, pravastatin, fluvastatin, atorvastatin, rosuvastatin or pitavastatin.

In a preferred embodiment of the invention, the compounds of the invention are administered in combination with a squalene synthesis inhibitor, by way of example and with preference BMS-188494, RPR 107393 or TAK-475.

In a preferred embodiment of the invention, the compounds of the invention are administered in combination with an ACAT inhibitor, by way of example and with preference avasimibe, melinamide, pactimibe, eflucimibe, lecimibide, CP-113818 or SMP-797.

In a preferred embodiment of the invention, the compounds of the invention are administered in combination with an MTP inhibitor, by way of example and with preference implitapide, BMS-201038, R-103757, CP346086, AEGR-733, LAB678 or JTT-130.

In a preferred embodiment of the invention, the compounds of the invention are administered in combination with a PPAR-gamma agonist, by way of example and with preference pioglitazone, ciglitazone or rosiglitazone.

In a preferred embodiment of the invention, the compounds of the invention are administered in combination with a PPAR-delta agonist, by way of example and with preference GW 501516 or BAY 68-5042.

In a preferred embodiment of the invention, the compounds of the invention are administered in combination with a cholesterol absorption inhibitor, by way of example and with preference ezetimibe, tiqueside, pamaqueside or colesevelam.

In a preferred embodiment of the invention, the compounds of the invention are administered in combination with a lipase inhibitor, by way of example and with preference orlistat.

In a preferred embodiment of the invention, the compounds of the invention are administered in combination with a polymeric bile acid adsorbent, by way of example and with preference cholestyramine, colestipol, colesolvam, CholestaGel or colestimide.

In a preferred embodiment of the invention, the compounds of the invention are administered in combination with a bile acid reabsorption inhibitor, by way of example and with preference ASBT (=IBAT) inhibitors, for example AZD-7806, S-8921, AK-105, BARI-1741, SC-435 or SC-635.

In a preferred embodiment of the invention, the compounds of the invention are administered in combination with a lipoprotein(a) antagonist, by way of example and with preference gemcabene calcium (CI-1027) or nicotinic acid.

In a preferred embodiment of the invention, the compounds of the invention are administered in combination with a calcium antagonist, by way of example and with preference nifedipine, amlodipine, nitrendipine, felodipine, lercanidipine, nimodipine, nicardipine, lacidipine, isradipine, nisoldipine, nilvadipine, manidipine, verapamil or diltiazem.

In a preferred embodiment of the invention, the compounds of the invention are administered in combination with an alpha-1-receptor blocker, by way of example and with preference prazosin, terazosin, doxazosin, trimazosin, and the first-generation unselective blockers phentolamine and phenoxybenzamine.

In a preferred embodiment of the invention, the compounds of the invention are administered in combination with a beta-receptor blocker, by way of example and with preference propranolol, atenolol, timolol, pindolol, alprenolol, oxprenolol, penbutolol, bupranolol, metipranolol, nadolol, mepindolol, carazolol, sotalol, metoprolol, betaxolol, celiprolol, bisoprolol, carteolol, esmolol, labetalol, carvedilol, adaprolol, landiolol, nebivolol, epanolol, acebutolol, betaxolol, pindolol, levibunolol or bucindolol.

In a preferred embodiment of the invention, the compounds of the invention are administered in combination with an angiotensin AT-1 receptor antagonist, by way of example and with preference losartan, candesartan, valsartan, telmisartan, irbesartan, eprosartan, olmesartan or embursatan.

In a preferred embodiment of the invention, the compounds of the invention are administered in combination with an ACE inhibitor, by way of example and with preference enalapril, captopril, lisinopril, spimpril, ramipril, delapril, fosinopril, quinopril, perindopril or trandopril.

In a preferred embodiment of the invention, the compounds of the invention are administered in combination with an endothelin antagonist, by way of example and with preference bosentan, darusentan, atrasentan, ambrisentan or sitaxsentan.

In a preferred embodiment of the invention, the compounds of the invention are administered in combination with a renin inhibitor, by way of example and with preference aliskiren, SPP-600, SPP-800, SPP-1148, VTP27999 or MK-8141.

In a preferred embodiment of the invention, the compounds of the invention are administered in combination with a mineralocorticoid receptor antagonist, by way of example and with preference spironolactone or eplerenone.

In a preferred embodiment of the invention, the compounds of the invention are administered in combination with a loop diuretic, by way of example and with preference bumetanide, ethaciynic acid, torasemide or furosemide; in combination with thiazides, by way of example and with preference chlorthiazide, chlorthalidone, hydrochlorthiazide, hydroflumethiazide, indapamide, methyclothiazide, metolazone or polythiazide; in combination with potassium-sparing diuretics, by way of example and with preference amiloride, eplerenone, spironolactone or triamterene and/or in combination with carbonic anhydrase inhibitors, by way of example and with preference acetazolamide, dichlophenamide or methazolamide.

In a preferred embodiment of the invention, the compounds of the invention are administered in combination with a vasopressin receptor antagonist, by way of example and with preference tolvaptan.

In a preferred embodiment of the invention, the compounds of the invention are administered in combination with a platelet aggregation inhibitor, by way of example and with preference aspirin, clopidogrel, ticlopidine, pmsugrel, tirofiban or dipyridamole.

In a preferred embodiment of the invention, the compounds of the invention are administered in combination with a thrombin inhibitor, by way of example and with preference ximelagatran, dabigatmn, melagatran, argatroban, bivalimdin, hirudin, lepimdin, desimdin or clexane.

In a preferred embodiment of the invention, the compounds of the invention are administered in combination with a GPIIb/IIIa antagonist, by way of example and with preference tirofiban or abciximab.

In a preferred embodiment of the invention, the compounds of the invention are administered in combination with a factor Xa inhibitor, by way of example and with preference rivaroxaban, DU-176b, apixaban, otamixaban, fidexaban, razaxaban, edoxaban, enoxaparin, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds of the invention are administered in combination with heparin or with a low molecular weight (LMW) heparin derivative.

In a preferred embodiment of the invention, the compounds of the invention are administered in combination with a vitamin K antagonist, by way of example and with preference warfarin, coumarin, acenocoumarol, phenprocoumon or dicumarol.

The present invention further provides medicaments which comprise at least one compound of the invention, typically together with one or more inert, nontoxic, pharmaceutically suitable excipients, and for the use thereof for the aforementioned purposes.

The compounds of the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds of the invention can be administered in administration forms suitable for these administration routes.

Suitable administration forms for oral administration are those which work according to the prior art and release the compounds of the invention rapidly and/or in a modified manner and which contain the compounds of the invention in crystalline and/or amorphized and/or dissolved form, for example tablets (uncoated or coated tablets, for example with gastric juice-resistant or retarded-dissolution or insoluble coatings which control the release of the compound of the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route) Administration forms suitable for parenteral administration include preparations for injection and infusion in the form of solutions, suspensions, emulsions, lyophilizates or sterile powders.

For the other administration routes, suitable examples are inhalable medicament forms (including powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Oral and parenteral administration are preferred, especially oral and intravenous administration.

The compounds of the invention can be converted to the administration forms mentioned. This can be accomplished in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

In general, it has been found to be advantageous in the case of parenteral administration to administer amounts of about 0.001 to 1 mg/kg, preferably about 0.01 to 0.5 mg/kg, of body weight to achieve effective results. In the case of oral administration the dosage is about 0.01 to 100 mg/kg, preferably about 0.01 to 20 mg/kg and most preferably 0.1 to 10 mg/kg of body weight.

It may nevertheless be necessary in some cases to deviate from the stated amounts, specifically as a function of body weight, route of administration, individual response to the active ingredient, nature of the preparation and time or interval over which administration takes place. Thus, in some cases less than the abovementioned minimum amount may be sufficient, while in other cases the upper limit mentioned must be exceeded. In the case of administration of greater amounts, it may be advisable to divide them into several individual doses over the day.

The working examples which follow illustrate the invention. The invention is not restricted to the examples.

All the compounds of the formula (I) according to the invention can be prepared by processes described in the prior art, for example in WO 03/053441 and WO 2010/086101.

### A. WORKING EXAMPLES

### Example 1

### 2-Amino-6-({[2-(4-chlorophenyl)-1,3-thiazol-4-yl]methyl}sulphanyl)-4-[4-(2-hydroxyethoxy)phenyl]pyridine-3,5-dicarbonitrile

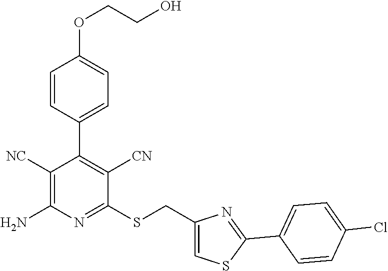 [SMILES: N#Cc1c(N)nc(SCc2csc(-c3ccc(Cl)cc3)n2)c(C#N)c1-c1ccc(OCCO)cc1]

The compound was prepared as described in WO 03/053441.

### Example 2

### 2-({[2-(4-Chlorophenyl)-1,3-thiazol-4-yl]methyl}sulphanyl)-6-(diethylamino)-4-[4-(2-hydroxyethoxy)phenyl]pyridine-3,5-dicarbonitrile

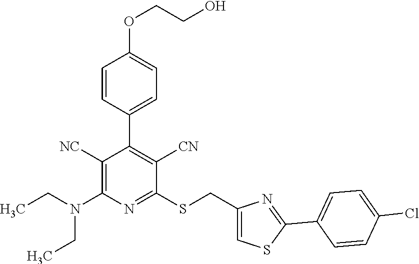 [SMILES: CCN(CC)c1nc(SCc2csc(-c3ccc(Cl)cc3)n2)c(C#N)c(-c2ccc(OCCO)cc2)c1C#N]

The compound was prepared as described in WO 2010/086101.

### Example 3

### 2-({[2-(4-Chlorophenyl)-1,3-thiazol-4-yl]methyl}sulphanyl)-4-[4-(2-hydroxyethoxy)phenyl]-6-(3-methoxyazetidin-1-yl)pyridine-3,5-dicarbonitrile

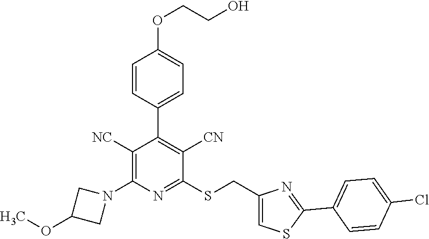 [SMILES: COC1CN(c2nc(SCc3csc(-c4ccc(Cl)cc4)n3)c(C#N)c(-c3ccc(OCCO)cc3)c2C#N)C1]

The compound was prepared as described in WO 2010/086101.

### Example 4

### 2-({[2-(4-Chlorophenyl)-1,3-thiazol-4-yl]methyl}sulphanyl)-4-[4-(2-hydroxyethoxy)phenyl]-6-(pyrrolidin-1-yl)pyridine-3,5-dicarbonitrile

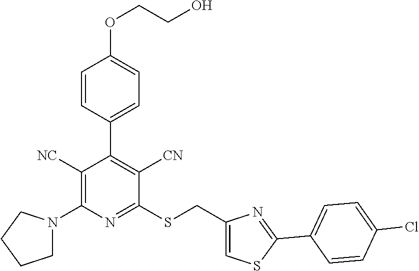 [SMILES: N#Cc1c(SCc2csc(-c3ccc(Cl)cc3)n2)nc(N2CCCC2)c(C#N)c1-c1ccc(OCCO)cc1]

The compound was prepared as described in WO 2010/086101.

### Example 5

### 2-({[2-(4-Chlorophenyl)-1,3-thiazol-4-yl]methyl}sulphanyl)-4-[4-(2-hydroxyethoxy)phenyl]-6-(piperidin-1-yl)pyridine-3,5-dicarbonitrile

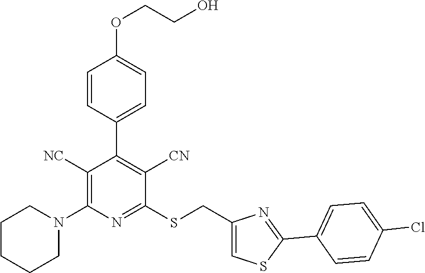 [SMILES: N#Cc1c(SCc2csc(-c3ccc(Cl)cc3)n2)nc(N2CCCCC2)c(C#N)c1-c1ccc(OCCO)cc1]

The compound was prepared as described in WO 2010/086101.

### Example 6

### 2-{4-[2-({[2-(4-Chlorophenyl)-1,3-thiazol-4-yl]methyl}sulfanyl)-3,5-dicyano-6-(pyrrolidin-1-yl)pyridin-4-yl]phenoxy}ethyl L-ornithinate bis(trifluoroacetate)

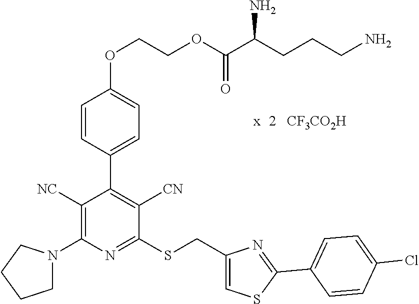 [SMILES: N#Cc1c(SCc2csc(-c3ccc(Cl)cc3)n2)nc(N2CCCC2)c(C#N)c1-c1ccc(OCCOC(=O)[C@@H](N)CCCN)cc1 ; "CF3CO2H"]

The compound was prepared as described in WO 2010/086101.

### Example 7

### 2-{4-[2-Amino-6-({[2-(4-chlorophenyl)-1,3-thiazol-4-yl]methyl}sulphanyl)-3,5-dicyanopyridin-4-yl]phenoxy}ethyl L-ornithinate dihydrochloride

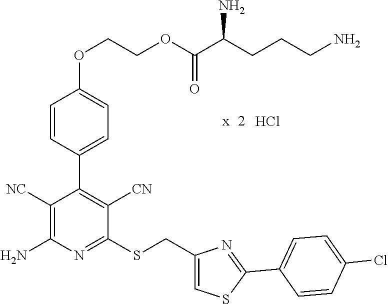 [SMILES: N#Cc1c(N)nc(SCc2csc(-c3ccc(Cl)cc3)n2)c(C#N)c1-c1ccc(OCCOC(=O)[C@@H](N)CCCN)cc1 ; Cl]

The compound was prepared as described in WO 2009/015812.

### Example 8

### 2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl-N-[(2S)-2,4-diaminobutanoyl] L-alaninate dihydrochloride

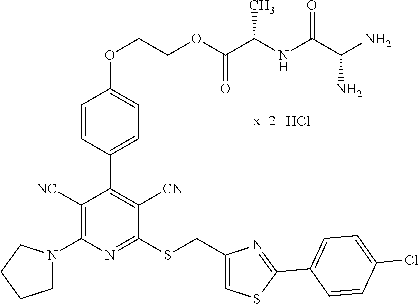

The compound was prepared as described in WO 2010/086101.

### Example 9

### 2-{4-[2-Amino-6-({[2-(4-chlorophenyl)-1,3-thiazol-4-yl]methyl}sulphanyl)-3,5-dicyanopyridin-4-yl]phenoxy}ethyl-N-[(2S)-2,4-diaminobutanoyl] L-alaninate dihydrochloride

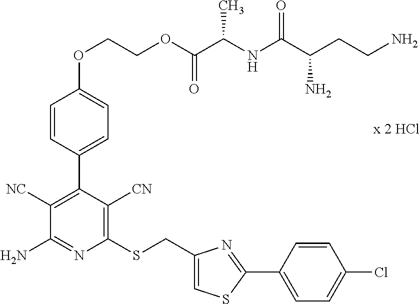 [SMILES: C[C@H](NC(=O)[C@@H](N)CCN)C(=O)OCCOc1ccc(-c2c(C#N)c(N)nc(SCc3csc(-c4ccc(Cl)cc4)n3)c2C#N)cc1]

The compound was prepared as described in WO 2009/015811.

### Example 10

### 2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl-L-lysyl L-alaninate dihydrochloride

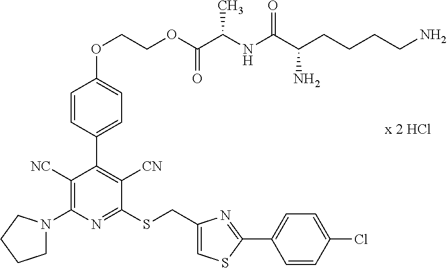 [SMILES: C[C@H](NC(=O)[C@@H](N)CCCCN)C(=O)OCCOc1ccc(-c2c(C#N)c(SCc3csc(-c4ccc(Cl)cc4)n3)nc(N3CCCC3)c2C#N)cc1]

The compound was prepared as described in WO 2010/086101.

### Example 11

### 2-{4-[2-Amino-6-({[2-(4-chlorophenyl)-1,3-thiazol-4-yl]methyl}sulphanyl)-3,5-dicyanopyridin-4-yl]phenoxy}ethyl-L-lysyl L-alaninate dihydrochloride

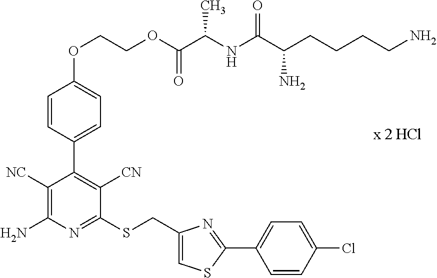 [SMILES: C[C@H](NC(=O)[C@@H](N)CCCCN)C(=O)OCCOc1ccc(-c2c(C#N)c(N)nc(SCc3csc(-c4ccc(Cl)cc4)n3)c2C#N)cc1]

The compound was prepared as described in WO 2009/015811.

### Example 12

### 2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl-L-alanyl L-alaninate hydrochloride

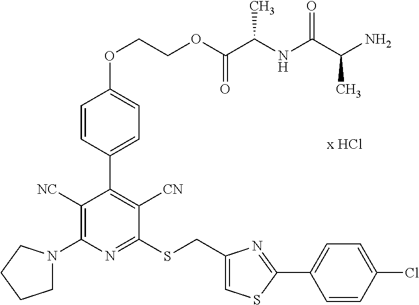 [SMILES: C[C@H](N)C(=O)N[C@@H](C)C(=O)OCCOc1ccc(-c2c(C#N)c(SCc3csc(-c4ccc(Cl)cc4)n3)nc(N3CCCC3)c2C#N)cc1]

The compound was prepared as described in WO 2010/086101.

### Example 13

### 2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl-L-argyl L-alaninate dihydrochloride

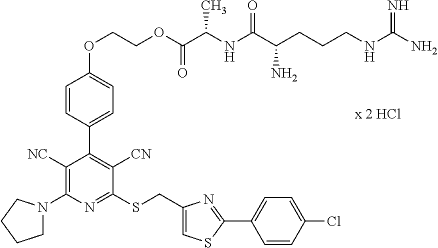 [SMILES: C[C@H](NC(=O)[C@@H](N)CCCNC(=N)N)C(=O)OCCOc1ccc(-c2c(C#N)c(SCc3csc(-c4ccc(Cl)cc4)n3)nc(N3CCCC3)c2C#N)cc1]

The compound was prepared as described in WO 2010/086101.

### Example 14

### 2-{4-[2-Amino-6-({[2-(4-chlorophenyl)-1,3-thiazol-4-yl]methyl}sulphanyl)-3,5-dicyanopyridin-4-yl]phenoxy}ethyl-L-argyl L-alaninate dihydrochloride

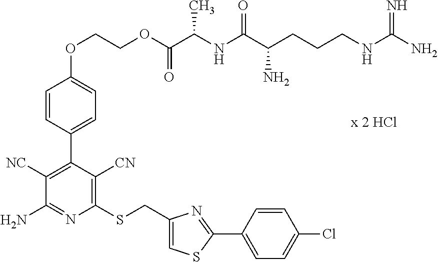 [SMILES: C[C@H](NC(=O)[C@@H](N)CCCNC(=N)N)C(=O)OCCOc1ccc(-c2c(C#N)c(N)nc(SCc3csc(-c4ccc(Cl)cc4)n3)c2C#N)cc1]

The compound was prepared as described in WO 2009/015811.

### Example 15

### 2-{4-[2-({[2-(4-Chlorophenyl)-1,3-thiazol-4-yl]methyl}sulphanyl)-3,5-dicyano-6-(pyrrolidin-1-yl)pyridin-4-yl]phenoxy}ethyl-L-histidyl L-alaninate dihydrochloride

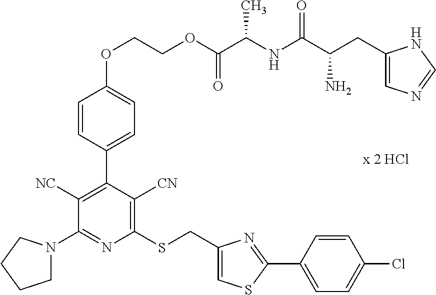 [SMILES: C[C@H](NC(=O)[C@@H](N)Cc1cnc[nH]1)C(=O)OCCOc1ccc(-c2c(C#N)c(SCc3csc(-c4ccc(Cl)cc4)n3)nc(N3CCCC3)c2C#N)cc1]

The compound was prepared as described in WO 2010/086101.

### Example 16

### 2-{4-[2-Amino-6-({[2-(4-chlorophenyl)-1,3-thiazol-4-yl]methyl}sulphanyl)-3,5-dicyanopyridin-4-yl]phenoxy}ethyl-L-histidyl L-alaninate dihydrochlorid

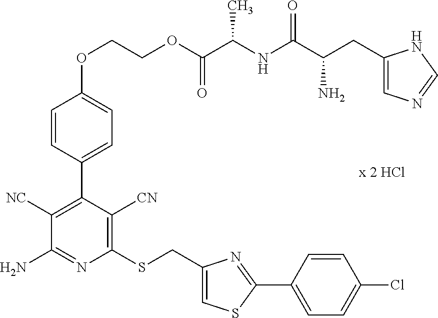 [SMILES: C[C@H](NC(=O)[C@@H](N)Cc1cnc[nH]1)C(=O)OCCOc1ccc(-c2c(C#N)c(N)nc(SCc3csc(-c4ccc(Cl)cc4)n3)c2C#N)cc1]

The compound was prepared as described in WO 2009/015811.

### B. ASSESSMENT OF PHARMACOLOGICAL AND PHYSIOLOGICAL EFFICACY

The pharmacological and physiological activity of the compounds of the invention can be demonstrated in the following assays:

B-1. Indirect Determination of Adenosine Agonistic Action Via Gene Expression

Cells of the CHO (Chinese Hamster Ovary) permanent line are stably transfected with the cDNA for adenosine receptor subtypes A1, A2a and A2b. The adenosine A1 receptors are coupled via G proteins and the adenosine A2a and A2b receptors via Gₛ proteins to adenylate cyclase. Correspondingly, cAMP formation in the cell is inhibited or stimulated By means of a cAMP-dependent promoter, expression of luciferase is then modulated. With the aim of high sensitivity and reproducibility, low variance and good suitability for carrying out on a robotic system, the luciferase assay is optimized by varying several test parameters, e.g. cell density, duration of primary culture and test incubation, forskolin concentration and composition of the medium. The following assay protocol is used for pharmacological characterization of the cells and for robot-assisted substance screening:

The stock cultures are grown in DMEM/F12 medium with 10% FCS (fetal calf serum) at 37° C. and 5% CO₂ and in each case are split 1:10 after 2-3 days. Test cultures are seeded in 384-well plates at 2000 cells per well and are cultured for approx. 48 hours at 37° C. Then the medium is replaced with a physiological saline solution (130 mM sodium chloride, 5 mM potassium chloride, 2 mM calcium chloride, 20 mM HEPES, 1 mM magnesium chloride hexahydrate, 5 mM sodium bicarbonate, pH 7.4). The test substances dissolved in DMSO are added by pipette in a dilution series from 5×10⁻¹¹ M to 3×10⁻⁶ M (final concentration) to the test cultures (maximum DMSO final concentration in the assay preparation: 0.5%). 10 minutes later, forskolin is added to the A1 cells and then all cultures are incubated for four hours at 37° C. Then 35 μl of a solution consisting of 50% of lysis reagent (30 mM disodium hydrogen phosphate, 10% glycerol, 3% TritonX100, 25 mM TrisHCl, 2 mM dithiothreitol (DTT), pH 7.8) and 50% of luciferase substrate solution (2.5 mM ATP, 0.5 mM luciferin, 0.1 mM coenzyme A, 10 mM tricine, 1.35 mM magnesium sulphate, 15 mM DTT, pH 7.8) is added to the test cultures, shaken for approx. 1 minute and the luciferase activity is measured with a camera system. The EC₅₀ values are determined, i.e. the concentrations at which, for the A1 cell, 50% of the luciferase response is inhibited or, for the A2b and A2a cells, 50% of the maximum capacity for stimulation is reached with the corresponding substance. The reference compound used in these experiments is the adenosine analogue NECA (5-N-ethylcarboxamido-adenosine), which binds with high affinity to all adenosine receptor subtypes and possesses agonistic action [Klotz, K. N., Hessling, J., Hegler, J., Owman, C, Kull, B., Fredholm, B. B., Lohse, M. J., “Comparative pharmacology of human adenosine receptor subtypes—characterization of stably transfected receptors in CHO cells”, Naunyn Schmiedebergs Arch. Pharmacol. 357, 1-9 (1998)].

The receptor selectivity and partiality can be determined from the action of the substances on cell lines that express the respective receptor subtypes after stable transfection with the corresponding cDNA (cf. the work by M. E. Olah, H. Ren, J. Ostrowski, K. A. Jacobson, G. L. Stiles, “Cloning, expression, and characterization of the unique bovine A1 adenosine receptor. Studies on the ligand binding site by site-directed mutagenesis”, J. Biol. Chem. 267 (1992), pages 10764-10770, the disclosure of which is hereby included in its entirety by reference).

The action of the substances on these cell lines can be detected by biochemical measurement of the intracellular messenger cAMP (cf. the work by K. N. Klotz, J. Hessling, J. Hegler, C. Owman, B Kull, B. B. Fredholm, M. J. Lohse, “Comparative pharmacology of human adenosine receptor subtypes—characterization of stably transfected receptors in CHO cells”, Naunyn Schmiedebergs Arch. Pharmacol. 357 (1998), pages 1-9, the disclosure of which is hereby included in its entirety by reference).

B-2. Acute Renal Protection by Partial A1 Agonists in the Glyeriol Rat (Haemolytic-Uraemic Syndrome; Example for Acute Kidney Disorders)

Glycerol-induced haemolysis in rats is an established animal model for investigating haemolysis-induced, acute renal failure with decrease of the individual nephron filtrate and a rapid increase in substances that are usually eliminated in the urine, such as creatinine and urea. Haemolytic uraemia leads to acute blockade of the tubules through protein deposits (“luminal casts”) and finally to degeneration/necrosis of the tubules. At present there is no known curative therapy, i.e. an antidegenerative therapy of structural kidney injury.

Procedure: male Wistar:WU rats (200-220 g) from Charles River received, after withdrawal of water for 14-18 hours, a single subcutaneous injection of a 50% glycerol solution (8 ml/kg body weight) or 0.9% NaCl (controls). The following groups were investigated:

Group 1 Controls; n=10

Group 2 Glycerol+placebo (60 g glycerol+100 g water+969 g PEG-400); n=12

Group 3 Glycerol+10 mg/kg BW partial A1 agonist EXAMPLE 1, p.o. in placebo, 8 and 19 hours after glycerol injection; n=8

At the end of the protocol (=24 hours after glycerol intoxication), the following samples were obtained/measurements were carried out: four-hour urine collection with administration of water (10 ml water/kg body weight, p.o.), and TaqMan analyses of renal injury markers in the renal cortex of the right kidney and histological assessment of the “kidney injury score” in the left kidney, by determining the degree of renal damage from the intensity of “luminal casts+tubular necrosis/degeneration”.

TABLE 3
Effects of the partial A1 agonist EXAMPLE 1 on functional
and structural dysfunction in the glycerol-rat model

| | | |
|---|---|---|
| Group 1 | Group 2 | Group 3 |

Plasma (P)

| | | | |
|---|---|---|---|
| P-creatinine | 47 ± 1** | 73 ± 15 | 52 ± 1* |
| [mmol/l] | | | |
| P-urea | 5 ± 0.2** | 10 ± 3.0 | 11 ± 0.4* |
| [mmol/l] | | | |

Urine (U)

| | | | |
|---|---|---|---|
| Urine volume | 2.9 ± 0.2 | 3.1 ± 0.5 | 6.7 ± 0.6*** |
| [ml/kg BW/h] | | | |
| U-creatinine | 1.6 ± 0.2 | 1.1 ± 0.2 | 0.2 ± 0.03*** |
| [μmol/ml urine/kg | | | |
| BW/h] | | | |
| U-urea | 148 ± 25 | 122 ± 20 | 31 ± 6*** |
| [μmol/ml urine/kg | | | |
| BW/h] | | | |
| U-sodium | 10 ± 3** | 20 ± 4 | 4 ± 1*** |
| [μmol/ml urine/kg | | | |
| BW/h] | | | |
| U-protein | 0.17 ± 0.03** | 0.52 ± 0.14 | 0.02 ± 0.01*** |
| [μmol/ml urine/kg | | | |
| BW/h] | | | |

Kidney injury markers

| | | | |
|---|---|---|---|
| Kidney injury | 15 ± 3*** | 3099 ± 866 | 951 ± 216** |
| marker-1 | | | |
| [rel. expression] | | | |
| Osteopontin | 1530 ± 143*** | 10596 ± 1859 | 5575 ± 694** |
| [rel. expression] | | | |
| Lipocalin-2 | 114 ± 7** | 787 ± 251 | 204 ± 23* |
| [rel. expression] | | | |
| Kidney injury score | 0.00 | 5.25 | 1.12 |
| (histo) | | | |

The values shown are mean values ± standard deviation. Statistical analysis comprised a one-way ANOVA with Newman-Keuls multivariance analysis with *p < 0.05, ***p < 0.01 and ***p < 0.001 vs. group 2 (GraphPad Prism 4.0).

The values shown are mean values±standard deviation. Statistical analysis comprised a one-way ANOVA with Newman-Keuls multivariance analysis with *p<0.05, ***p<0.01 and ***p<0.001 vs. group 2 (GraphPad Prism 4.0).

B-3. Effects of Partial A1 Agonists on Renal and Cardiac Function in 5/6 Nephrectomized Rats (Example 1 for Chronic Kidney Disorder)

5/6 nephrectomy in the rat, in which one kidney and the renal cortex of the second kidney are surgically removed (=5/6 of functional kidney tissue) is an established animal model for interstitial renal fibrosis and chronic renal insufficiency [C. Fleck et al., “Suitability of 5/6 nephrectomy (5/6NX) for the induction of interstitial renal fibrosis in rats Influence of sex, strain, and surgical procedure”, Exp. Tox. Pathology 2006, 57, 195-205]. The model is characterized by progressive proteinuria, disturbed tubular electrolyte transport, a reduced glomerular filtration rate, an increase in urinary output, interstitial fibrosis with reactively occurring lymphocyte infiltrates, glomerulosclerosis and tubular atrophy. The level of proteinuria is a specific index of the renal function prognosis: the higher the proteinuria, the greater is the functional and structural kidney injury and therefore progression of chronic to terminal renal insufficiency (uraemia). During monitoring of therapy, e.g. with the ACE inhibitor enalapril, the course of proteinuria provides information on the functional and structural response to the therapy and thus on the renal function prognosis. As a result of the renal insufficiency and accumulation of umemic toxins, in the animals there is secondary development of a umemic cardiomyopathy, depending on the severity associated with hypertension, increased heart rate, reduced parasympathetic tone, myocardial, left-ventricular hypertrophy and fibrosis, diastolic dysfunction and an increased prevalence of arrhythmias [Svilerova et al. 2010 Physiol Res 59: S81-S88).

Procedure: male Wistar:WU rats (210-250 g) from Charles River had the left kidney and the renal cortex of the right kidney removed (=SNX) in one session; sham-operated rats served as control group (=SOP). The following groups, each with 12 rats per group, were investigated:

Group 1 SOP

Group 2 SNX+placebo (60 g glycerol+100 g water+969 g PEG-400)

Group 3 SNX+10 mg/kg body weight/day enalapril (reference therapy, in the drinking water)

Group 4 SNX+0.1 mg/kg body weight/day partial A1 agonist EXAMPLE 1, p.o. in placebo

Group 5 SNX+1.0 mg/kg body weight/day partial A1 agonist EXAMPLE 1, p.o. in placebo

Group 6 SNX+0.3 mg/kg body weight/day partial A1 agonist EXAMPLE 4, p.o. in placebo

Group 7 SNX+3.0 mg/kg body weight/day partial A1 agonist EXAMPLE 4, p.o. in placebo

Three weeks after 5/6 nephrectomy, the rats developed manifest proteinuria, from this time point the rats were treated for a further four weeks corresponding to the groups stated above. At the end of the protocol, the following samples were obtained/measurements were carried out: eight-hour urine collection with determination of the concentration of hFABP in the urine by ELISA (Rat/Mouse h-FABP ELISA, Hycultbiotech), as marker for end organ damage, invasive measurement of the isoprenaline-induced increase (cumulatively 12.5 ng-250 ng/kg isoprenaline, i.v.) in systolic and diastolic left ventricular function, and TaqMan analyses of renal injury markers in the renal cortex.

Results:

TABLE 1
Effects of the partial A1 agonists EXAMPLE 1 and EXAMPLE 4 on functional and structural renal
dysfunction in the 5/6 nephrectomy rat model compared with enalapril as reference therapy

| | | | | | | |
|---|---|---|---|---|---|---|
| Group 1 | Group 2 | Group 3 | Group 4 | Group 5 | Group 6 | Group 7 |

Plasma (P)

| | | | | | | | |
|---|---|---|---|---|---|---|---|
| P-creatinine | 50 ± 1*** | 65 ± 1 | 70 ± 2** | 66 ± 1 | 63 ± 1 | 64 ± 1 | 63 ± 1 |
| [mmol/l] | | | | | | | |
| P-urea | 7 ± 0.2*** | 10 ± 0.3 | 15 ± 1*** | 11 ± 0.5 | 11 ± 0.3 | 11 ± 0.3 | 11 ± 0.3 |
| [mmol/l] | | | | | | | |

Urine (U)

| | | | | | | | |
|---|---|---|---|---|---|---|---|
| Urine volume | 1.2 ± 0.1** | 1.8 ± 0.1 | 2.8 ± 0.2*** | 2.2 ± 0.1 | 2.0 ± 0.2 | 1.8 ± 0.1 | 1.8 ± 0.2 |
| [ml/kg BW/h] | | | | | | | |
| U-creatinine | 8.6 ± 1.7*** | 3.3 ± 0.2 | 1.6 ± 0.2** | 2.4 ± 0.3 | 2.3 ± 0.3 | 3.1 ± 0.2 | 2.7 ± 0.3 |
| [μmol/ml urine/ | | | | | | | |
| kg BW/h] | | | | | | | |
| U-urea [μmol/ml | 818 ± 162*** | 349 ± 29 | 198 ± 22** | 260 ± 31 | 264 ± 35 | 330 ± 25 | 281 ± 30 |
| urine/kg BW/h] | | | | | | | |
| U-sodium | 38 ± 9* | 17 ± 2 | 18 ± 3 | 18 ± 3 | 31 ± 3 | 19 ± 3 | 43 ± 5** |
| [μmol/ml urine/ | | | | | | | |
| kg BW/h] | | | | | | | |
| U-protein | 1.14 ± 0.25* | 2.19 ± 0.51 | 0.25 ± 0.04*** | 1.07 ± 0.28* | 0.75 ± 0.14** | 1.70 ± 0.29 | 0.97 ± 0.12* |
| [μmol/ml urine/ | | | | | | | |
| kg BW/h] | | | | | | | |

Kidney injury markers [gene expression]

| | | | | | | | |
|---|---|---|---|---|---|---|---|
| Kidney injury | 11 ± 4*** | 182 ± 54 | 27 ± 17*** | 60 ± 17** | 62 ± 13** | 68 ± 14** | 56 ± 18** |
| marker-1 [rel. | | | | | | | |
| expression] | | | | | | | |
| Osteopontin [rel. | 1653 ± 143*** | 16243 ± 4343 | 6226 ± 4107* | 6008 ± 1209* | 5476 ± 1131** | 6882 ± 1240* | 6340 ± 1656** |
| expression] | | | | | | | |
| Lipocalin-2 [rel. | 169 ± 11** | 887 ± 222 | 400 ± 236* | 307 ± 51* | 313 ± 65* | 340 ± 49* | 283 ± 45* |
| expression] | | | | | | | |

End organ injury marker concentration in the urine

| | | | | | | | |
|---|---|---|---|---|---|---|---|
| pg hFABP/ml | 414 ± 291*** | 4726 ± 1792 | 377 ± 151*** | 1088 ± 159** | 1136 ± 229** | 3125 ± 497 | 1706 ± 249* |
| urine/h | | | | | | | |
| pg hFABP/μmol | 22 ± 15*** | 515 ± 186 | 83 ± 33*** | 194 ± 36*** | 175 ± 39*** | 386 ± 57 | 218 ± 39** |
| creatinine | | | | | | | |

The values shown are mean values ± standard deviation from 10-12 measurements.
Statistical analysis carried out with a one-way ANOVA with Newman-Keuls multivariance analysis with *p < 0.05, ***p < 0.01 and ***p < 0.001 vs. group 2 (GraphPad Prism 4.0).

TABLE 2
Effects of the partial A1 agonists EXAMPLE 1 and EXAMPLE 4 on left-ventricular diastolic dysfunction,
measured as hyperresponsive left-ventricular pressure decrease as a function of time (in mmHg/s) as a result
of isoprenaline infusion [ng/kg body weight], in the 5/6 nephrectomy rat model compared with enalapril as
reference therapy

| | Group 1 | Group 2 | Group 3 | Group 4 | Group 5 | Group 6 | Group 7 |
|---|---|---|---|---|---|---|---|
| Isoprenaline | (n = 10) | (n = 10) | (n = 12) | (n = 12) | (n = 12) | (n = 12) | (n = 10) |
| 0 | 7230 ± 316 | 7029 ± 298 | 6613 ± 226 | 6769 ± 206 | 6516 ± 334 | 6518 ± 221 | 6884 ± 383 |
| 12.5 | 8383 ± 367 | 8449 ± 351 | 7972 ± 280 | 7990 ± 208 | 7126 ± 283 | 7733 ± 233 | 8369 ± 395 |
| 25 | 8453 ± 364 | 8653 ± 308 | 8297 ± 285 | 8283 ± 222 | 7309 ± 285 | 8001 ± 224 | 8557 ± 396 |
| 50 | 8909 ± 339 | 9310 ± 276 | 8705 ± 227 | 8913 ± 282 | 7990 ± 213 | 8763 ± 195 | 9100 ± 341 |
| 100 | 9581 ± 352 | 10108 ± 364 | 9267 ± 177 | 9701 ± 300 | 8722 ± 183 | 9621 ± 212 | 10263 ± 294 |
| 250 | 9737 ± 372*** | 10259 ± 351 | 9359 ± 202*** | 9721 ± 307*** | 9028 ± 226*** | 9968 ± 162*** | 10385 ± 352 |

The values shown are mean values ± standard deviation.
Statistical analysis comprised a one-way ANOVA with Bonferroni multivariance analysis and the variables isoprenaline dose vs. therapy, with ***p < 0.001 versus group 2 over the whole dose-effect curve (GraphPad Prism 4.0).

B-4. Effects of Partial A1 Agonists on Renal and Cardiac Function in Unilaterally Nephrectomized and Spontaneously Hypertensive Rats (Example 2 for Chronic Kidney Disorders)

Unilaterally nephrectomized, spontaneously hypertensive rats (SHR) are an established animal model for investigating the development and progression of hypertensive damage of the glomeruli in the remaining kidney [H. Kinuno et al., “Effects of uninephrectomy on renal structural properties in spontaneously hypertensive rats”, Clin and Exp Pharmacol and Physiol 2005, 32, 173-178]. This damage is characterized by increased intraglomerular pressure and intraglomerular flow with simultaneously reduced glomerular filtration rate. Furthermore, renal excretion of substances usually eliminated in the urine, such as creatinine and urea, is reduced and the electrolyte metabolism is disturbed, the latter being associated with volume retention.

Procedure: male SHR rats (180-220 g) from Taconic had the left kidney removed in one session (=UNX). Normotensive Wistar Kyoto rats (WKY) from Taconic served as control. The following groups, each with 12 rats per group, were investigated:

Group 1 WKY

Group 2 UNX+placebo (60 g glycerol+100 g water+969 g PEG-400)

Group 3 UNX+10 mg/kg body weight/day enalapril (reference therapy, in the drinking water)

Group 4 SNX+1.0 mg/kg body weight/day partial A1 EXAMPLE 1, p.o. in placebo

Group 5 SNX+3.0 mg/kg body weight/day partial A1 EXAMPLE 4, p.o. in placebo

Three weeks after unilateral nephrectomy, the SHRs developed manifest hypertensive damage of the glomeruli in the remaining kidney, from this time point on the rats were treated for a further four weeks corresponding to the groups given above. At the end of the protocol, the following samples were obtained/measurements were carried out: eight-hour urine collection, invasive measurement of systolic and diastolic left ventricular function, and TaqMan analyses of renal injury markers in the renal cortex.

TABLE 4
Effects of the partial A1 agonists EXAMPLE 1 and EXAMPLE 4 on functional and structural renal
dysfunction in unilaterally nephrectomized SHR compared with enalapril as reference therapy

| | | | | |
|---|---|---|---|---|
| Group 1 | Group 2 | Group 3 | Group 4 | Group 5 |

Plasma (P)

| | | | | | |
|---|---|---|---|---|---|
| P-creatinine | 50 ± 1.2* | 56 ± 0.8 | 62 ± 1.6* | 62 ± 1.1* | 58 ± 0.6 |
| [mmol/l] | | | | | |
| P-urea | 7.0 ± 0.5** | 9.1 ± 0.4 | 9.3 ± 0.4 | 9.3 ± 0.4 | 8.7 ± 0.2 |
| [mmol/l] | | | | | |

Urine (U)

| | | | | | |
|---|---|---|---|---|---|
| Urine volume | 1.9 ± 0.3 | 1.7 ± 0.3 | 2.4 ± 0.2 | 2.1 ± 0.2 | 1.8 ± 0.2 |
| [ml/kg BW/h] | | | | | |
| U-creatinine | 2.9 ± 0.6 | 4.3 ± 1.1 | 1.5 ± 0.3** | 2.1 ± 0.4* | 3.4 ± 0.9 |
| [μmol/ml urine/kg BW/h] | | | | | |
| U-urea | 306 ± 65 | 400 ± 105 | 153 ± 27** | 186 ± 31* | 337 ± 89 |
| [μmol/ml urine/kg BW/h] | | | | | |
| U-sodium | 41 ± 8 | 29 ± 9 | 30 ± 5 | 37 ± 9 | 46 ± 11 |
| [μmol/ml urine/kg BW/h] | | | | | |
| U-protein | 0.68 ± 0.15* | 1.23 ± 0.34 | 0.43 ± 0.07** | 0.53 ± 0.10* | 0.96 ± 0.26 |
| [μmol/ml urine/kg BW/h] | | | | | |

Kidney injury markers [gene expression]

| | | | | | |
|---|---|---|---|---|---|
| CYR61 | 278 ± 32*** | 656 ± 73 | 460 ± 55* | 395 ± 47** | 274 ± 21*** |
| [rel. expression] | | | | | |
| Lipocalin-2 | 53 ± 3*** | 433 ± 239 | 140 ± 23* | 160 ± 24* | 119 ± 9** |
| [rel. expression] | | | | | |

The values shown are mean values ± standard deviation. Statistical analysis comprised a one-way ANOVA with Newman-Keuls multivariance analysis with *p < 0.05, ***p < 0.01 and ***p < 0.001 vs. group 2 (GraphPad Prism 4.0).

TABLE 5
Effects of the partial A1 agonists EXAMPLE 1 and EXAMPLE 4 on functional and structural myocardial
dysfunction in unilaterally nephrectomized SHR compared with enalapril as reference therapy

| | Group 1 | Group 2 | Group 3 | Group 4 | Group 5 |
|---|---|---|---|---|---|
| Heart rate beats/min | 318 ± 10 | 352 ± 10 | 353 ± 7 | 295 ± 11*** | 326 ± 9 |
| Systolic blood pressure mmHg | 117 ± 4*** | 209 ± 3 | 132 ± 4*** | 171 ± 8*** | 205 ± 5 |
| Diastolic blood pressure mmHg | 89 ± 4*** | 151 ± 5 | 91 ± 5*** | 112 ± 6*** | 144 ± 6 |
| Left ventricular hypertrophy | 3.3 ± 0.1*** | 4.9 ± 0.2 | 3.7 ± 0.2*** | 4.3 ± 0.2 | 4.5 ± 0.2 |
| (LVS/RV ratio, mg/mg) | | | | | |
| Left-ventricular pressure mmHg | 111 ± 3*** | 198 ± 5 | 130 ± 5*** | 169 ± 8** | 196 ± 5 |
| Left-ventricular end-diastolic | 8.1 ± 0.5 | 11.5 ± 1.1 | 5.4 ± 0.6 | 8.5 ± 1.3 | 10.22 ± 1.6 |
| pressure mmHg | | | | | |
| Pressure increase over time | 6656 ± 379*** | 9923 ± 356 | 8114 ± 304* | 7943 ± 512** | 9419 ± 383 |
| (in mmHg/s) | | | | | |
| Pressure decrease over time | −7108 ± 646*** | −11362 ± 327 | −10130 ± 239* | −10181 ± 523 | −10680 ± 524 |
| (in mmHg/s) | | | | | |

The values shown are mean values ± standard deviation. Statistical analysis comprised a one-way ANOVA with Newman-Keuls multivariance analysis with *p < 0.05, ***p < 0.01 and ***p < 0.001 vs. group 2 (GraphPad Prism 4.0).

B-5. Determination of Pharmacokinetic Parameters Following Intravenous and Oral Administration

The test substance is administered to animals (e g mouse, rat, dog) intravenously as solution; it is administered orally as solution or suspension by stomach tube. After administration of the substance, blood is obtained from the animals at fixed time points. It is heparinized, then plasma is obtained from it by centrifugation. The substance is quantified analytically in the plasma by LC/MS-MS. From the plasma concentration-time curves thus determined, the pharmacokinetic parameters such as AUC (area under the concentration-time curve), Cmax (peak plasma concentration), T1/2 (half-life) and CL (clearance) are calculated by means of a validated pharmacokinetic computer program.

B-6. Determination of the Plasma Free Fraction by Means of Transil

The distributions of a compound between water and surface-supported egg-lecithin membranes (Transil) on the one hand (MA_{buffer}) and between plasma and surface-supported egg-lecithin membranes (Transil) on the other hand (MAₚₗₐₛₘₐ) are measured.

The dissolved test substance is added by pipette to suspensions of Transil/buffer and Transil/plasma. After these incubations, the Transil is separated by centrifugation at 1800 g from the respective phase. The concentrations of substance before centrifugation and in the supernatant after centrifugation are determined. The free fraction is calculated as the ratio of the membrane affinity in plasma (MAₚₗₐₛₘₐ) and in buffer (MA_{buffer}).

B-7. CNS Effects of Substances

Possible effects of a single oral administration of a test substance on behavioral parameters, movement activity (“open field test”) and body temperature are investigated in rats. The test substances are administered orally in increasing dosage. Control animals receive only the vehicle (ethanol/Solutol/water (10:40:50, v/v/v). Each treatment group consists of 6 male rats. The animals are examined for behavioral changes and changes in body temperature after 0.5, 1, 2, and 7 hours. After approx. 0.5 and 7 hours the animals are also examined for possible substance-dependent changes in their movement activity in the “open field test” (free movement in the cage). Plasma concentrations of the test substances are determined in satellite groups.

### C. WORKING EXAMPLES OF PHARMACEUTICAL COMPOSITIONS

The compounds of the invention can be converted to pharmaceutical formulations as follows:

Tablet:

Composition:

100 mg of the compound of the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate. Tablet weight 212 mg. Diameter 8 mm, radius of curvature 12 mm.

Production:

The mixture of compound of the invention, lactose and starch is granulated with a 5% solution (w/w) of the PVP in water. The granules are dried and then mixed with the magnesium stearate for 5 minutes. This mixture is compressed in a conventional tabletting press (see above for format of the tablet). The guide value used for the pressing is a pressing force of 15 kN.

Suspension for Oral Administration:

Composition:

1000 mg of the compound of the invention, 1000 mg of ethanol (96%), 400 mg of Rhodigel® (xanthan gum from FMC, Pennsylvania, USA) and 99 g of water. 10 ml of oral suspension correspond to a single dose of 100 mg of the compound of the invention.

Production:

The Rhodigel is suspended in ethanol; the compound of the invention is added to the suspension. The water is added while stirring. The mixture is stirred for about 6 h before swelling of the Rhodigel is complete.

Solution for Oral Administration:

Composition:

500 mg of the compound of the invention, 2.5 g of polysorbate and 97 g of polyethylene glycol 400. 20 g of oral solution correspond to a single dose of 100 mg of the compound of the invention.

Production:

The compound of the invention is suspended in the mixture of polyethylene glycol and polysorbate with stirring. The stirring operation is continued until dissolution of the compound of the invention is complete.

i.v. solution:

The compound of the invention is dissolved in a concentration below the saturation solubility in a physiologically acceptable solvent (e.g. isotonic saline solution, glucose solution 5% and/or PEG 400 solution 30%). The solution is subjected to sterile filtration and dispensed into sterile and pyrogen-free injection vessels.

1. A compound of the formula (I) 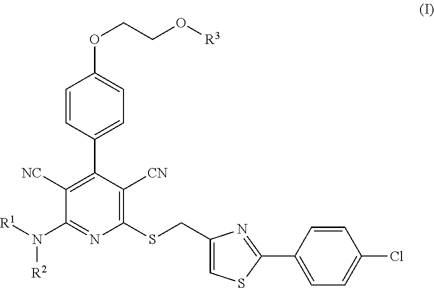
in which
R¹ represents hydrogen or (C₁-C₄)-alkyl,
R² represents hydrogen or (C₁-C₄)-alkyl,
where (C₁-C₄)-alkyl may be substituted by 1 to 3 substituents independently of one another selected from the group consisting of fluorine, trifluoromethyl, trifluoromethoxy, (C₁-C₄)-alkoxy, (C₃-C₇)-cycloalkyl, (C₃-C₇)-cycloalkoxy and (C₁-C₄)-alkyl sulphonyl,
or
R¹ and R² together with the nitrogen atom to which they are attached form a 4- to 7-membered heterocycle which may contain a further heteroatom from the group consisting of N, O and S,
where the 4- to 7-membered heterocycle may be substituted by 1 to 2 substituents independently of one another selected from the group consisting of fluorine, trifluoromethyl, (C₁-C₄)-alkyl, trifluoromethoxy and (C₁-C₄)-alkoxy,
R³ represents hydrogen or a group of the formula 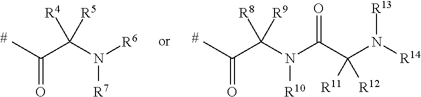
where
# represents the point of attachment to the oxygen atom,
R⁴ represents hydrogen or the side group of a natural α-amino acid or its homologues or isomers,
R⁵ represents hydrogen,
R⁶ represents hydrogen,
R⁷ represents hydrogen,
R⁸ represents hydrogen or the side group of a natural α-amino acid or its homologues or isomers,
R⁹ represents hydrogen,
R¹⁰ represents hydrogen or methyl,
R¹¹ represents hydrogen or the side group of a natural α-amino acid or its homologues or isomers,
R¹² represents hydrogen,
R¹³ represents hydrogen,
R¹⁴ represents hydrogen,
and the N-oxides, salts, solvates, salts of the N-oxides and solvates of the N-oxides or salts thereof,
wherein the compound is for use in a method for the treatment and/or prevention of acute and/or chronic kidney disorders.

2. The compound of claim 1 of the formula (I) in which
R¹ represents (C₁-C₄)-alkyl,
R² represents (C₁-C₄)-alkyl,
where (C₁-C₄)-alkyl may be substituted by 1 to 3 substituents independently of one another selected from the group consisting of fluorine, trifluoromethyl, trifluoromethoxy, (C₁-C₄)-alkoxy, (C₃-C₇)-cycloalkyl, (C₃-C₇)-cycloalkoxy and (C₁-C₄)-alkyl sulphonyl,
or
R¹ and R² are each hydrogen
or
R¹ and R² together with the nitrogen atom to which they are attached form a 4- to 7-membered heterocycle which may contain a further heteroatom from the group consisting of N, O and S,
where the 4- to 7-membered heterocycle may be substituted by 1 to 2 substituents independently of one another selected from the group consisting of fluorine, trifluoromethyl, (C₁-C₄)-alkyl, trifluoromethoxy and (C₁-C₄)-alkoxy,
R³ represents hydrogen or a group of the formula 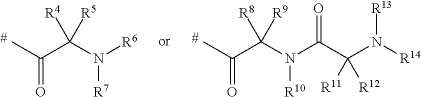
where
# represents the point of attachment to the oxygen atom,
R⁴ represents hydrogen or the side group of a natural α-amino acid or its homologues or isomers,
R⁵ represents hydrogen,
R⁶ represents hydrogen,
R⁷ represents hydrogen,
R⁸ represents hydrogen or the side group of a natural α-amino acid or its homologues or isomers,
R⁹ represents hydrogen,
R¹⁰ represents hydrogen or methyl,
R¹¹ represents hydrogen or the side group of a natural α-amino acid or its homologues or isomers,
R¹² represents hydrogen,
R¹³ represents hydrogen,
and the N-oxides, salts, solvates, salts of the N-oxides and solvates of the N-oxides or salts thereof,
wherein the compound is for use in a method for the treatment and/or prevention of acute and/or chronic kidney disorders.

3. The compound of claim 1 of the formula (I) in which
R¹ represents hydrogen or (C₁-C₃)-alkyl,
R² represents hydrogen or (C₁-C₃)-alkyl,
where (C₁-C₃)-alkyl may be substituted by 1 or 2 substituents independently of one another selected from the group consisting of fluorine, trifluoromethyl, methoxy, ethoxy, cyclopropyl and cyclobutyl,
R¹ and R² together with the nitrogen atom to which they are attached form a 4- to 6-membered heterocycle which may contain a further heteroatom from the group consisting of N, O and S,
where the 4- to 6-membered heterocycle may be substituted by 1 to 2 substituents independently of one another selected from the group consisting of fluorine, trifluoromethyl, (C₁-C₄)-alkyl, trifluoromethoxy, methoxy and ethoxy,
R³ represents hydrogen or a group of the formula 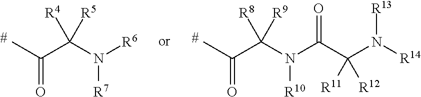
where
# represents the point of attachment to the oxygen atom,
R⁴ represents 3-aminopropan-1-yl,
R⁵ represents hydrogen,
R⁶ represents hydrogen,
R⁷ represents hydrogen,
R⁸ represents methyl,
R⁹ represents hydrogen,
R¹⁰ represents hydrogen,
R¹¹ represents methyl, 2-aminoeth-1-yl, 4-aminobut-1-yl, 3-guanidinopropan-1-yl or imidazol-4-ylmethyl,
R¹² represents hydrogen,
R¹³ represents hydrogen,
R¹⁴ represents hydrogen,
and the N-oxides, salts, solvates, salts of the N-oxides and solvates of the N-oxides or salts thereof,
wherein the compound is for use in a method for the treatment and/or prevention of acute and/or chronic kidney disorders.

4. The compound of claim 1 of the formula (I) in which
R¹ represents hydrogen or ethyl,
R² represents hydrogen or ethyl,
or
R¹ and R² together with the nitrogen atom to which they are attached form a 4- to 6-membered heterocycle which may contain a further heteroatom from the group consisting of N, O and S,
R³ represents hydrogen or a group of the formula 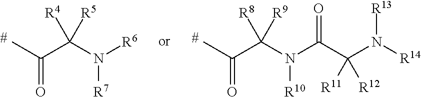
where
# represents the point of attachment to the oxygen atom,
R⁴ represents 3-aminopropan-1-yl,
R⁵ represents hydrogen,
R⁶ represents hydrogen,
R⁷ represents hydrogen,
R⁸ represents methyl,
R⁹ represents hydrogen,
R¹⁰ represents hydrogen,
R¹¹ represents methyl, 2-aminoeth-1-yl, 4-aminobut-1-yl, 3-guanidinopropan-1-yl or imidazol-4-ylmethyl,
R¹² represents hydrogen,
R¹³ represents hydrogen,
R¹⁴ represents hydrogen,
and the N-oxides, salts, solvates, salts of the N-oxides and solvates of the N-oxides or salts thereof,
wherein the compound is for use in a method for the treatment and/or prevention of acute and/or chronic kidney disorders.

5. The compound of claim 1 of the formula (I) in which
R¹ represents hydrogen or ethyl,
R² represents hydrogen or ethyl,
or
R¹ and R² together with the nitrogen atom to which they are attached form an azetidinyl, pyrrolidinyl or piperidinyl ring,
where the azetidinyl ring may be substituted by a methoxy substituent,
R³ represents hydrogen or a group of the formula 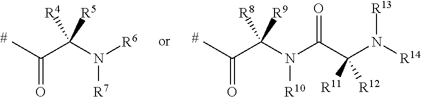
where
# represents the point of attachment to the oxygen atom,
R⁴ represents 3-aminopropan-1-yl,
R⁵ represents hydrogen,
R⁶ represents hydrogen,
R⁷ represents hydrogen,
R⁸ represents methyl,
R⁹ represents hydrogen,
R¹⁰ represents hydrogen,
R¹¹ represents methyl, 2-aminoeth-1-yl, 4-aminobut-1-yl, 3-guanidinopropan-1-yl or imidazol-4-ylmethyl,
R¹² represents hydrogen,
R¹³ represents hydrogen,
R¹⁴ represents hydrogen,
and the N-oxides, salts, solvates, salts of the N-oxides and solvates of the N-oxides or salts thereof,
wherein the compound is for use in a method for the treatment and/or prevention of acute and/or chronic kidney disorders.

6. The compound of claim 1 of the formula (I) selected from the group below:
2-amino-6-({[2-(4-chlorophenyl)-1,3-thiazol-4-yl]methyl}sulphanyl)-4-[4-(2-hydroxyethoxy)phenyl]pyridine-3,5-dicarbonitrile
2-({[2-(4-chlorophenyl)-1,3-thiazol-4-yl]methyl}sulphanyl)-6-(di ethyl amino)-4-[4-(2-hydroxyethoxy)phenyl]pyridine-3,5-dicarbonitrile
2-({[2-(4-chlorophenyl)-1,3-thiazol-4-yl]methyl}sulphanyl)-4-[4-(2-hydroxyethoxy)phenyl]-6-(3-methoxyazetidin-1-yl)pyridine-3,5-dicarbonitrile
2-({[2-(4-chlorophenyl)-1,3-thiazol-4-yl]methyl}sulphanyl)-4-[4-(2-hydroxyethoxy)phenyl]-6-(pyrrolidin-1-yl)pyridine-3,5-dicarbonitrile
2-({[2-(4-chlorophenyl)-1,3-thiazol-4-yl]methyl}sulphanyl)-4-[4-(2-hydroxyethoxy)phenyl]-6-(piperidin-1-yl)pyridine-3,5-dicarbonitrile,
and the N-oxides, salts, solvates, salts of the N-oxides and solvates of the N-oxides or salts thereof,
wherein the compound is for use in a method for the treatment and/or prevention of acute and/or chronic kidney disorders.

7. A compound of the formula 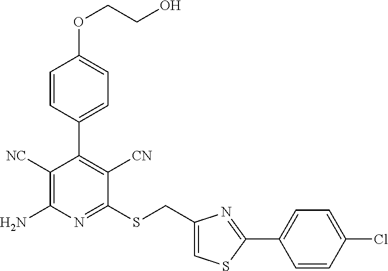 [SMILES: N#Cc1c(N)nc(SCc2csc(-c3ccc(Cl)cc3)n2)c(C#N)c1-c1ccc(OCCO)cc1]
2-amino-6-({[2-(4-chlorophenyl)-1,3-thiazol-4-yl]methyl}sulphanyl)-4-[4-(2-hydroxyethoxy)phenyl]pyridine-3,5-dicarbonitrile
and the N-oxides, salts, solvates, salts of the N-oxides and solvates of the N-oxides or salts thereof,
wherein the compound is for use in a method for the treatment and/or prevention of acute and/or chronic kidney disorders.

8. A method for treating and/or preventing acute and/or chronic kidney disorders resulting from renocardiac and/or cardiorenal syndrome in humans and animals, using an effective amount of at least one compound of formula (I), as defined in claim 1, or of a medicinal product comprising at least one compound of formula (I) as defined in claim 1 in combination with an inert, nontoxic pharmaceutically suitable additive.

9. A medicament comprising the compound of claim 1, in combination with an inert, nontoxic, pharmaceutically suitable additive.

10. A method of making a medicament comprising combining the compound of claim 1 with an inert, nontoxic, pharmaceutically suitable additive.

11. The compound of claim 1, wherein the method for the treatment and/or prevention of acute and/or chronic kidney disorders is with concomitant acute and/or chronic heart disorders owing to renocardiac and/or cardiorenal syndrome.
